(19) United States
(12) Patent Application Publication
(10) Pub. No.: US 2026/0199217 A1
(43) Pub. Date: Jul. 16, 2026

(54) BCMA-SPECIFIC ANTIBODY CONSTRUCTS AND COMPOSITIONS THEREOF

(71) Applicant: L'OREAL, PARIS (FR)

(72) Inventors: Jeremy M. KINDER, Seattle, WA (US); Neal VAN HOEVEN, Seattle, WA (US)

(21) Appl. No.: 19/136,551

(22) PCT Filed: Dec. 8, 2023
(86) PCT No.: PCT/US2023/083053

§ 371 Date: Jun. 6, 2025

Related U.S. Application Data
(60) Provisional application No. 63/386,836, filed on Dec. 9, 2022.

(51) Int. Cl. A61K 8/73; A61K 8/25; A61K 8/49; A61K 8/67; A61K 8/87; A61Q 19/08

(52) U.S. Cl. CPC A61K 8/73; A61K 8/25; A61K 8/4953; A61K 8/675; A61K 8/676; A61K 8/678; A61K 8/87; A61Q 19/08; A61K 2800/95

(57) ABSTRACT

Disclosed herein are antibodies or antigen binding fragments thereof that specifically bind human BCMA. Also disclosed are chimeric antigen receptors and chimeric antigen receptor transgenes comprising an antigen binding domain that specifically binds human BCMA. Also described herein are immune cells, viral vectors, and other compositions containing the antibodies, antibody binding fragments, chimeric antigen receptors and/or chimeric antigen receptor transgenes.

### CROSS-REFERENCE TO RELATED APPLICATIONS

This application claims benefit of U.S. Ser. No. 63/477,913, filed Dec. 30, 2022, and benefit of French Application No. FR 2303024, filed on Mar. 29, 2023, which are incorporated herein by reference in their entirety.

### FIELD OF THE DISCLOSURE

The present disclosure relates to skin perfecting and tightening compositions for application to the skin. The compositions provide an instantaneous and dramatic improvement to the appearance of skin, for example, by reducing the appearance of wrinkles, eye bags, pores, and skin imperfections such as scarring, dark spots (and uneven skin tone), dark circles, and roughness.

### BACKGROUND

Skin produces less collagen and elastin as it ages. For example, after the age of twenty, a person (human) produces about 1 percent less collagen in the skin each year. As a result, the skin becomes thinner and more fragile. Inevitably, wrinkles, crow's feet, age-spots, eye bags, and the like, begin to form. In addition, many individuals suffer from scarring or other aberrations in the skin. Consumers often wish to improve the appearance of such age-related skin imperfections, preferably with instantaneous results. Many consumer products and procedures devoted to hiding and reducing wrinkles are available. Some products and procedures are simple and inexpensive, for example, applying make-up, particularly a primer or colored foundation, to cover the skin (and thereby cover and/or fill the wrinkles and provide a smoother look). Far more expensive and drastic procedures, such as surgical face lifts and Botox® injections, are also used to reduce the appearance of wrinkles. However, many consumers either cannot afford, or do not wish, to undergo such drastic cosmetic procedures. There are several lotions and creams that are formulated to hydrate the skin and make it more supple, thereby reducing the appearance of wrinkles. Some of these products contain active ingredients, for example, niacinamide, that help repair and rejuvenate skin over time. Unfortunately, many of these types of products suffer from various drawbacks.

Make-up products are often visible, offer minimal texture benefits, and have no long-lasting effect on the skin. After removal of the make-up, the skin looks the same as before the make-up was applied. Common skin care products can have chronic, acute or both effects on the skin. Hydration and optical effects are common acute benefits, but these benefits quickly wear-off over time.

Attempts have been made to develop new categories of products to improve the appearance of skin without the drawbacks of existing products and procedures. One such family of products can be generally classified as “adhesive, contractile film-forming polymers.” Film-forming polymers are chemical compositions that when applied to skin, leave a pliable, cohesive and continuous covering. A select group of film-forming polymers are also adhesive to the skin and contractile.

### SUMMARY OF THE DISCLOSURE

The instant disclosure relates to skin perfecting and tightening compositions for providing an instantaneous and dramatic improvement to the appearance of skin, e.g., by reducing the appearance of wrinkles, eye bags, pores, and skin imperfections such as scarring, dark spots (and uneven skin tone), dark circles, and roughness. Upon application to the skin, the compositions immediately begin tightening the skin while maintaining the skin's natural appearance, i.e., the treated skin does not appear to be manipulated or modified. The inventors discovered that the skin perfecting and tightening compositions exhibit excellent transparency, haze, and gloss characteristics appropriate for application to the skin, which are long lasting. The skin perfecting and tightening compositions typically include:
(a) pullulan;
(b) optionally, one or more hydrophilic non-acrylate film-forming polymers other than pullulan;
(c) one or more mineral clays;
(d) one or more particulate fillers;
(e) optionally, one or more water-soluble organic solvents; and
(f) water.

In various embodiments, the skin perfecting and tightening compositions may additionally include: (g) one or more fibers, (h) one or more skin active agents, and/or (i) one or more miscellaneous ingredients.

Nonlimiting examples of hydrophilic non-acrylate film-forming polymers other than pullulan include polyurethanes, vinyl polymers, natural polymers, latex or pseudolatex polymers, vinylpyrrolidone-based polymers, amphoteric polymers, or a combination thereof. More specific yet nonlimiting examples of hydrophilic non-acrylate film-forming polymers include polysaccharides and polysaccharide derivatives (e.g., carrageenan and dehydroxyxanthan gum), polyurethanes, polyvinyl alcohol, sodium hyaluronate, vinyl polymers, natural polymers, copolymers derived from C₄-C₈ monounsaturated carboxylic acids or anhydrides, methyl vinyl ether/butyl monomaleate copolymers, polyvinyl pyrollidone, polyvinyl acetate, water soluble gums, water soluble celluloses, dextrans, hyaluronic acid, cyclodextrins, polyvinyl caprolactam-polyvinyl acetate-polyethylene glycol graft copolymers, or a mixture thereof. In various embodiments, at least one of the one or more hydrophilic non-acrylate film-forming polymers other than pullulan is a polyurethane, for example, polyurethane-99 and/or polyurethane-35.

Nonlimiting examples of mineral clays include montmorilonite, hectorite, saponite, bidelite, kaolinite, montmorillonites, saponites, laponites, hectorites (including disteardimonium hectorite), smectites, illites, vermiculite, mica, and synthetic mica.

Nonlimiting examples of particulate fillers include carnauba wax, fluorphlogopite, titanium dioxide, tin oxide, sodium acrylates crosspolymer-2, cellulose, perlite, silica, perlite, silica silylate, talc, mica, titanated mica, alumina, aluminum silicate, silica which may or may not be coated, fumed silica, polyamide, methyl methacrylate crosspolymer, poly(methyl (meth)acrylate), polyethylene powder, polymethylsilsesquioxane powder, nylon-12, cellulose, nylon-12, polyvinylalcohol crosspolymer, or a mixture thereof.

In various embodiments, the skin perfecting and tightening composition includes one or more fibers (or filaments). Nonlimiting examples of fibers (or filaments) include non-woven fibers, wood pulp fibers, cotton fibers, collagen fibers, modified collagen fibers, chitin fibers, modified chitin fibers, chitosan fibers, modified chitosan fibers, polysaccharide fibers, modified polysaccharide fibers, protein fibers, hydrolyzed protein fibers, fibrin fibers, silk protein fibers, silk fibrin fibers, peptide fibers, cellulose fibers, modified cellulose fibers, bio-cellulose fibers, acetic bio-cellulose fibers, bacterial cellulose fibers, chondroitin sulfate fibers, sodium alginate fivers, agar fibers, natural fibers, alginic acid fibers, cocoon filament and combination thereof.

One or more water-soluble organic solvents may optionally be included in the skin perfecting and tightening compositions. Nonlimiting examples of water-soluble organic solvents include glycerin, mono-alcohols, polyols (polyhydric alcohols), glycols, and a mixture thereof. More specific but nonlimiting examples of water-soluble organic solvents include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples are ethylene glycol, propylene glycol, butylene glycol, hexylene glycol, propane diol, and glycerin.

The skin perfecting and tightening compositions may optionally include one or more fibers (or filaments). Nonlimiting examples of fibers (or filaments) include non-woven fibers, wood pulp fibers, cotton fibers, collagen fibers, modified collagen fibers, chitin fibers, modified chitin fibers, chitosan fibers, modified chitosan fibers, polysaccharide fibers, modified polysaccharide fibers, protein fibers, hydrolyzed protein fibers, fibrin fibers, silk protein fibers, silk fibrin fibers, peptide fibers, cellulose fibers, modified cellulose fibers, bio-cellulose fibers, acetic bio-cellulose fibers, bacterial cellulose fibers, chondroitin sulfate fibers, sodium alginate fivers, agar fibers, natural fibers, alginic acid fibers, cocoon filament and combination thereof.

The skin perfecting and tightening compositions may optionally include one or more skin active agents. Nonlimiting examples (categories) of skin active agents include antioxidants, anti-wrinkle agents, depigmenting agents, vitamins, ceramides, alpha and/or beta hydroxy acids, anti-acne agents, etc. More specific but nonlimiting examples of skin active agents include vitamin A, vitamin C, vitamin D, vitamin E), niacinamide, caffeine, ceramides, ferulic acid, salicylic acid, madecassoside, retinoic acid, benzoyl peroxide, or a combination thereof.

The skin perfecting and tightening compositions may optionally include one or more miscellaneous ingredients. Nonlimiting examples include preservatives, fragrances, pH adjusters, salts, buffers, flavonoids, botanical extracts, UV filtering agents, proteins, protein hydrolysates and/or isolates, hydrotropes, pearlescent agents, fillers, colorants, mattifying agents, or a combination thereof.

The form of the skin perfecting and tightening composition is typically a liquid or liquid-like consistency, a gel or gel-like consistency, a lotion or a lotion-like consistency, a cream or a cream-like consistency, or a paste or a paste-like consistency. The form or consistency of the skin perfecting and tightening composition is such that it can be applied evenly over the skin. In various embodiments, the skin perfecting and tightening composition is in the form of an oil-in-water emulsion, a suspension, or a dispersion.

The instant disclosure also relates to methods for improving the appearance of skin comprising applying the compositions described herein to the skin. For example, the instant disclosure relates to methods for smoothing the skin comprising applying the compositions described herein to the skin and forming a film or layer on the skin. In various embodiments, the instant disclosure relates to methods for tightening the skin comprising applying the compositions described herein to the skin and forming a film or layer on the skin. In some instances, the compositions are applied to the skin of the face, and/or more specifically around the eyes, around the mouth, and/or around the neck of a human face. The methods of improving the appearance of skin include methods for treating or reducing the appearance of wrinkles, blemishes, dryness, roughness, dullness, age spots, scarring, divots, etc.

The compositions of the present disclosure are surprisingly stable, elastic, and provide an unexpectedly long-lasting skin smoothing and tightening effect. Unlike other products, the film or layer formed on the skin does not dry-out and whiten, crack, or peel. Instead, the film or layer remains flexible (elastic), durable, and comfortable. Moreover, the compositions (and resulting films or layers) hydrate and protect the underlying skin.

### DETAILED DESCRIPTION OF THE DISCLOSURE

The present disclosure relates to skin perfecting and tightening compositions that provide instantaneous and long-lasting improvements to the appearance of skin. For example, the compositions provide an instantaneous smoothing and tightening effect to skin and are therefore useful for treating eye bags, facial wrinkles, and other age-related skin imperfections. The skin perfecting and tightening compositions are also particularly useful for filling and improving skin aberrations such as deep wrinkles, divots in the skin, scarring of the skin, etc. The skin perfecting and tightening compositions typically include:
(a) about 1 to about 15 wt. % of pullulan;
(b) optionally, up to about 12 wt. % of one or more hydrophilic non-acrylate film-forming polymers other than pullulan, preferably polyurethane-99;
(c) about 1 to about 10 wt. % of one or more mineral clays, preferably laponite;
(d) about 1 to about 10 wt. % of one or more particulate fillers; and
(e) optionally, one or more water-soluble organic solvents; and
(f) about 60 to about 90 wt. % water;
   wherein all weight percentages are based on a total weight of the composition.

In various embodiments, the skin perfecting and tightening compositions may additionally include: (g) one or more fibers, (h) one or more skin active agents, and/or (i) one or more miscellaneous ingredients. Furthermore, the skin perfecting compositions may optionally be free or essentially free from acrylate and/or methacrylate polymers and copolymers (e.g., sodium polyacrylate, acrylates copolymer, acrylates/isobornyl acrylate copolymer, C₁-C₄ alkyl(methacrylate) polymers) regardless of what other monomer units appear in the polymer and copolymer (and crosspolymer).

### (a) Pullulan

Pullulan is a polysaccharide polymer consisting of maltotriose units, also known as α-1,4-; α-1,6-glucan′. Three glucose units in maltotriose are connected by an α-1,4 glycosidic bond, whereas consecutive maltotriose units are connected to each other by an α-1,6 glycosidic bond. The amount of pullulan in the skin perfecting and tightening compositions will vary. Nonetheless, in various embodiments, the skin perfecting and tightening compositions include about 1 to about 15 wt. % of pullulan, based on a total weight of the skin perfecting and tightening composition. In further embodiments, the skin perfecting and tightening composition includes about 1 to about 15 wt. %, about 1 to about 12 wt. %, about 1 to about 10 wt. %, about 1 to about 8 wt. %, about 2 to about 15 wt. %, about 2 to about 12 wt. %, about 2 to about 10 wt. %, about 2 to about 8 wt. %, about 3 to about 15 wt. %, about 3 to about 12 wt. %, about 3 to about 10 wt. %, about 3 to about 8 wt. % of pullulan, based on a total weight of the skin perfecting and tightening composition.

### (b) Hydrophilic Non-Acrylate Film-Forming Polymer

The term “hydrophilic non-acrylate film-forming polymer” is interchangeable with the term “aqueous non-acrylate film-forming polymer” and refers to non-acrylate polymers or copolymers that can be water-soluble or water-dispersible; anionic, cationic, or nonionic; and crosslinked or not cross-linked. They generally include polar groups such as hydroxyl, amide, amine or ether groups, and other groups with a high affinity for water. In addition, they should provide good adhesive, binding, film-forming and water-swelling properties. The hydrophilic non-acrylate polymers are film-forming polymers capable of forming, by themselves or in the presence of an auxiliary film-forming agent, a continuous film capable of adhering to a support, especially to the skin.

As the name indicates, hydrophilic non-acrylate film-forming polymers do not include acrylates (or methacrylates) and are not formed from acrylic acid monomers (or methacrylic acid monomers), regardless of whether the polymers are homopolymers, copolymers, or crosspolymers. For example, the hydrophilic non-acrylate film-forming polymer is not sodium polyacrylate (homopolymer), acrylates copolymer (copolymer), styrene acrylates copolymer (copolymer), nor acrylates/C10-30 alkyl acrylate crosspolymer (crosspolymer).

Nonlimiting examples of hydrophilic non-acrylate film forming polymers include polyurethanes, vinyl polymers, natural polymers, latex polymers, vinylpyrrolidone (VP)-based polymers, amphoteric polymers, and mixtures thereof.

### (i) Polyurethanes

The polyurethanes may be aliphatic, cycloaliphatic, or aromatic polyurethanes, polyurea-urethanes or polyurea copolymers, comprising, alone or as a mixture: at least one block of aliphatic and/or cycloaliphatic and/or aromatic polyester origin, and/or at least one branched or unbranched silicone block, for example polydimethylsiloxane or polymethylphenylsiloxane, and/or at least one block comprising fluoro groups.

The film-forming polyurethanes that can be used in the invention may also be obtained from branched or non-branched polyesters or from alkyls comprising labile hydrogens, which are modified by reaction with a diisocyanate and a difunctional organic compound (for example dihydroxy, diamino or hydroxyamino), also comprising either a carboxylic acid or carboxylate group, or a sulfonic acid or sulfonate group, or alternatively a neutralizable tertiary amine group or a quaternary ammonium group.

With a view to forming the polyurethane, monomers bearing an anionic group that can be used during the polycondensation that may be mentioned include dimethylolpropionic acid, trimellitic acid or a derivative such as trimellitic anhydride, the sodium salt of 3-sulfopentanediol acid, and the sodium salt of 5-sulfo-1,3-benzenedicarboxylic acid. Preferably, the monomer bearing an anionic group is dimethylolpropionic acid.

As film-forming polyurethane that may be used according to the invention, mention may thus be made of the aqueous polyurethane dispersions sold under the names AVALURE UR-405®, AVALURE UR-410®, AVALURE UR-425® and AVALURE UR-450@ by the company Goodrich. A particularly preferred polyurethane is polyurethane-99.

Advantageously, the film-forming polyurethanes that are selected from copolymers obtained by copolymerization of hexanediol, neopentyl glycol, adipic acid, hexamethylene diisocyanate, N-(2-aminoethyl)-3-aminoethanesulfonic acid and ethylenediamine. Preferably, the polyurethanes may also be selected from copolymers obtained by copolymerization of adipic acid, dicyclohexylmethane diisocyanate, ethylenediamine, hexanediol, neopentyl glycol and sodium N-(2-aminoethyl)-3-aminoethanesulfonate.

In particular, the polyurethanes are chosen from those sold under the name BAYCUSAN ECO E 1001, BAYCUSAN C1001 or C1004, known as polyurethane-99, polyurethane-35, and more particularly the product sold under the name BAYCUSAN C1001, known as polyurethane-99.

### (ii) Vinyl Polymers

Vinyl polymers may be chosen from polyvinyl alcohols, copolymers derived from C₄-C₈ monounsaturated carboxylic acids or anhydrides, and methyl vinyl ether/butyl monomaleate copolymers. For the purpose of the present invention, the term “polyvinyl alcohol” means a polymer comprising —CH₂CH(OH)— units. The polyvinyl alcohols are generally produced by hydrolysis of polyvinyl acetate. Usually, the reaction takes place in the presence of methanol (alcoholysis). The reaction is normally catalyzed by acidic or basic catalysis. The degree of hydrolysis of the commercial products is variable, often around 87%, but products with a 100 degree of hydrolysis also exist. Copolymers with monomers other than vinyl acetate also exist, such as ethylene/vinyl alcohol copolymers.

The polyvinyl alcohol polymers are preferably chosen from homopolymers or copolymers with vinyl acetate, the latter corresponding in particular to a partial hydrolysis of polyvinyl acetate.

Use may, for example, be made of the products of the CELVOL range provided by the company Celanese under the names CELVOL 540, CELVOL 350, CELVOL 325, CELVOL 165, CELVOL 125, CELVOL 540 S, CELVOL 840 and CELVOL 443.

The copolymer(s) derived from C₄-C₈ monounsaturated carboxylic acids or anhydrides may be chosen from copolymers comprising (i) one or more maleic, fumaric or itaconic acids or anhydrides and (ii) one or more monomers chosen from vinyl esters, vinyl ethers, vinyl halides, phenylvinyl derivatives, and acrylic acid and its esters, the anhydride functions of these copolymers being optionally monoesterified or monoamidated, for example, INCI name: Butyl Ester of PVM/MA Copolymer. Preferably, the copolymer(s) derived from C₄-C₈ monounsaturated carboxylic acids or anhydrides are chosen from the monoesterified methyl vinyl ether/maleic anhydride copolymers, for example, ethyl ester of PVM/MA copolymer, sold under the name GANTREZ ES 225 by the company ISP.

(iii) Natural Polymers

The hydrophilic polymers may also be chosen from natural polymers, in particular polysaccharides which have monosaccharides or disaccharides as base units. The natural polymers are preferably chosen from pullulan, guar gums and modified guar gums, celluloses, and gellan gum, and derivatives thereof. Guar gums are galactomannans consisting of mannose and galactose. For the purpose of the present disclosure, the term “modified guar gum” means guar gums alkylated with at least one C₁-C₈ alkyl group, guar gums hydroxyalkylated with at least one C₁₋₈ hydroxyalkyl group and guar gums acylated with at least one C₁₋₈ acyl group. Hydroxypropylated guar gums (e.g., hydroxypropyl guar) such as the product sold under the name JAGUAR HP 105 by the company Rhodia is a useful example.

The cellulose is a β1-4-polyacetal of cellobiose, cellobiose being a disaccharide consisting of two glucose molecules. The cellulose derivatives may be cationic, amphoteric or nonionic. Among these derivatives, cellulose ethers, cellulose esters and cellulose ester ethers are distinguished. Among the nonionic cellulose ethers, mention may be made of alkylcelluloses such as methylcelluloses and ethylcelluloses; hydroxyalkylcelluloses such as hydroxymethylcelluloses, hydroxyethylcelluloses and hydroxypropylcelluloses; and mixed hydroxyalkyl-alkylcelluloses such as hydroxypropylmethylcelluloses, hydroxyethylmethylcelluloses, hydroxyethylethylcelluloses and hydroxybutylmethylcelluloses.

Among the cationic cellulose ethers, mention may be made of crosslinked or non-crosslinked quaternized hydroxyethylcelluloses. The quaternizing agent may especially be glycidyltrimethylammonium chloride or a fatty amine such as laurylamine or stearylamine. Another cationic cellulose ether that may be mentioned is hydroxyethylcellulosehydroxypropyltrimethylammonium. Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates.

Among the cellulose ester ethers, mention may be made of hydroxypropylmethylcellulose phthalates and ethylcellulose sulfates. The cellulose-based compounds of the invention may be chosen from unsubstituted celluloses and substituted celluloses.

The celluloses and derivatives are represented, for example, by the products sold under the names AVICEL® (microcrystalline cellulose, MCC) by the company FMC Biopolymers, under the name METHOCEL™ (cellulose ethers) and ETHOCEL™ (ethylcellulose) by the company Dow, BENECEL® (methylcellulose), BLANOSE™ (carboxymethylcellulose), CULMINAI® (methylcellulose, hydroxypropylmethylcellulose), KLUCEL® (hydroxypropylcellulose), POLYSURF® (cetylhydroxyethylcellulose) and NATROSOL® CS (hydroxyethylcellulose) by the company Hercules Aqualon.

Gellan gum is a polysaccharide produced by aerobic fermentation of Sphingomonas elodea, more commonly known as Pseudomonas elodea. This linear polysaccharide is formed from the sequence of the following monosaccharides: D-glucose, D-glucuronic acid and L-rhamnose. In native form, gellan gum is highly acylated. The gellan gum preferably used in the film according to the present invention is a gellan gum that is at least partially deacylated. This at least partially deacylated gellan gum is obtained by high-temperature alkaline treatment. A solution of KOH or of NaOH will, for example, be used. The purified gellan gum sold under the trade name KELCOGEL® by the company Kelco is suitable for preparing the compositions according to the invention.

Gellan gum derivatives are all the products obtained by performing standard chemical reactions, especially such as esterifications, addition of a salt of an organic or mineral acid. Welan gum is used, for example, as a gellan gum derivative. Welan gum is a gellan gum modified by fermentation by means of Alcaligenes strain ATCC 31 555. Welan gum has a recurring pentasaccharide structure formed from a main chain consisting of D-glucose, D-glucuronic acid and L-rhamnose units, onto which a pendent L-rhamnose or L-mannose unit is grafted. The welan gum (diutan gum) sold under the trade name KELCO CRETE® by the company Kelco is suitable for preparing the compositions according to the invention.

As other saccharide polymers that can be used according to the invention, mention may be made of starches and derivatives thereof.

Natural hydrophilic film-forming polymers include celluloses and derivatives thereof, in particular those sold under the name AVICEL® (microcrystalline cellulose, MCC) by the company FMC Biopolymers.

Carrageenans are anionic polysaccharides constituting the cell walls of various red algae (Rhodophyceae) belonging to the Gigartinacae, Hypneaceae, Furcellariaceae and Polyideaceae families. They are generally obtained by hot aqueous extraction from natural strains of said algae. These linear polymers, formed by disaccharide units, are composed of two D-galactopyranose units linked alternately by α(1,3) and β(1,4) bonds. They are highly sulfated polysaccharides (20%-50%) and the α-D-galactopyranosyl residues may be in 3,6-anhydro form. Depending on the number and position of sulfate-ester groups on the repeating disaccharide of the molecule, several types of carrageenans are distinguished, namely: kappa-carrageenans, which bear one sulfate-ester group, iota-carrageenans, which bear two sulfate-ester groups, and lambda-carrageenans, which bear three sulfate-ester groups. Carrageenans are composed essentially of potassium, sodium, magnesium, triethanolamine and/or calcium salts of polysaccharide sulfate esters.

Carrageenans are sold especially by the company SEPPIC under the name SOLAGUM®, by the company Gelymar under the names CARRAGEL®, CARRALACT® and Carrasol®, by the company Cargill under the names Satiagel™ and SATIAGUM™, and by the company CP-Kelco under the names GENULACTA®, GENUGEL® and GENUVISCO®.

Mention is made of hyaluronic acid and salts therefore, for example sodium hyaluronate and potassium hyaluronate. Sodium hyaluronate is the sodium salt of hyaluronic acid. It is a glycosaminoglycan and long-chain polymer of disaccharide units of Na-glucuronate-N-acetylglucosamine.

A useful hydrophilic film forming polymer is xanthan gum and modified xanthan gums, such as dehydroxanthan gum, hydroxypropyl xanthan gum, and mixtures thereof. In some cases, dehydroxanthan gum is useful.

### (iv) Latex or Pseudolatex Polymers

As presented above for the hydrophilic film-forming polyurethanes, the hydrophilic film-forming polymer may thus also be present in a composition of the invention in the form of particles dispersed in an aqueous phase, which is generally known as a latex or pseudolatex. Techniques for preparing these dispersions are well known to those skilled in the art.

Aqueous dispersions of film-forming polymers that may be used include the acrylic dispersions sold under the names NEOCRYL XK-90, NEOCRYL A-1070®, NEOCRYL A-1090®, NEOCRYL BT-62®, NEOCRYL A-1079® and NEOCRYL A-523® by the company Avecia-Neoresins, Dow Latex 432® by the company Dow Chemical, DAITOSOL 5000 ADR or DAITOSOL 5000 SJ® by the company Daito Kasey Kogyo; SYNTRAN 5760® or SYNTRAN PC 5100® by the company Interpolymer, Allianz OPT by the company Röhm & Haas, aqueous dispersions of acrylic or styrene/acrylic polymers sold under the brand name JONCRYL® by the company Johnson Polymer, or the aqueous dispersions of polyurethane sold under the names NEOREZ R-981® and NEOREZ R-974@ by the company Avecia-Neoresins, AVALURE UR-405®, AVALURE UR-410®, AVALURE UR-425®, AVALURE UR-450®, SANCURE 875, SANCURE 861®, SANCURE 878® and SANCURE 2060® by the company Goodrich, IMPRANIL 85@ by the company Bayer and AQUAMERE H-1511® by the company Hydromer; the sulfopolyesters sold under the brand name EASTMAN AQR by the company Eastman Chemical Products, and vinyl dispersions, for instance MEXOMER PAM® from the company Chimex, and mixtures thereof.

### (v) Vinylpyrrolidone (VP)-Based Polymers

Nonlimiting examples of vinylpyrrolidone (VP)-based film-forming polymers include polyvinylpyrrolidone (PVP), VP-styrene copolymer, VP-vinyl acetate copolymer, and diethyl sulfate VP-dimethylaminoethyl-methacrylic acid copolymer, and blends thereof.

### (vi) Amphoteric Polymers

Nonlimiting examples of hydrophilic non-acrylic film-forming polymers include amphoteric polymers. Nonlimiting examples include polymethacryloyloxyethyltrimethyl ammonium chloride, alkyl vinyl ether maleic anhydride (AVE/MA) copolymer, diallyldimethylammonium chloride-maleamic acid copolymers, or a combination thereof.

The amount of the one or more hydrophilic non-acrylate film-forming polymers in the skin perfecting and tightening compositions will vary. Nonetheless, in various embodiments, the skin perfecting and tightening composition includes about 0.1 to about 12 wt. % of the one or more hydrophilic non-acrylic film-forming polymers, based on a total weight of the skin perfecting and tightening composition. In further embodiments, the skin perfecting and tightening composition includes about 0.1 to about 10 wt. %, about 0.1 to about 8 wt. %, about 0.1 to about 6 wt. %, 0.1 to about 5 wt. %, about 0.5 to about 12 wt. %, about 0.5 to about 10 wt. %, about 0.5 to about 8 wt. %, about 0.5 to about 6 wt. %, about 0.5 to about 5 wt. %, about 1 to about 12 wt. %, about 1 to about 10 wt. %, about 1 to about 8 wt. %, about 1 to about 5 wt. %, about 2 to about 12 wt. %, about 2 to about 10 wt. %, about 2 to about 8 wt. %, about 2 to about 6 wt. %, about 2 to about 5 wt. %, about 3 to about 12 wt. %, about 3 to about 10 wt. %, about 3 to about 8 wt. %, about 3 to about 6 wt. %, about 4 to about 12 wt. %, about 4 to about 10 wt. %, about 4 to about 8 wt. %, about 5 to about 8 wt. % of the one or more hydrophilic non-acrylate film-forming polymers based on a total weight of the skin perfecting and tightening composition.

### (c) Mineral Clays

Mineral clays include hydrous aluminium phyllosilicates (e.g. kaolin, Al₂Si₂O₅(OH)₄), sometimes with variable amounts of iron, magnesium, alkali metals, alkaline earths, and other cations. The term mineral clay is generally applied to (1) a natural material with plastic properties, (2) particles of very fine size, customarily those defined as particles smaller than two micrometres (7.9×10-5 inch), and (3) very fine mineral fragments or particles composed mostly of hydrous-layer silicates of aluminum, though occasionally containing magnesium and iron. Although, in a broader sense, mineral clays can include virtually any mineral of the above-cited particle size, the definition adapted here includes hydrous-layer silicates and some related short-range ordered aluminosilicates, both of which occur either exclusively or frequently in very fine-size grades.

Clay minerals are composed essentially of silica, alumina or magnesia or both, and water, but iron substitutes for aluminum and magnesium in varying degrees, and appreciable quantities of potassium, sodium, and calcium are frequently present as well. Some clay minerals may be expressed using ideal chemical formulas as the following: 2SiO₂·Al₂O₃·2H₂O (kaolinite), 4SiO₂·Al₂O₃·H₂O (pyrophyllithe), 4SiO₂·3MgO·H₂O (talc), and 3SiO₂·Al₂O₃·5FeO·4H₂O (chamosite). The SiO₂ ratio in a formula is the key factor determining clay mineral types. These minerals can be classified on the basis of variations of chemical composition and atomic structure into nine groups: (1) kaolin-serpentine (kaolinite, halloysite, lizardite, chrysotile), (2) pyrophyllite-talc, (3) mica (illite, glauconite, celadonite), (4) vermiculite, (5) smectite (montmorillonite, nontronite, saponite), (6) chlorite (sudoite, clinochlore, chamosite), (7) sepiolite-palygorskite, (8) interstratified clay minerals (e.g., rectorite, corrensite, tosudite), and (9) allophane-imogolite.

In various embodiments, the skin perfecting and tightening compositions of the instant disclosure do not require sodium silicate. Sodium silicate can certainly be included in the compositions but is not necessarily required. Accordingly, in various embodiments, the skin perfecting and tightening compositions are free or essentially free from sodium silicate. Likewise, in various embodiments, the skin perfecting and tightening compositions are free or essentially free from sodium silicate, magnesium silicate, calcium silicate, and/or aluminum silicate. In other embodiments the skin perfecting and tightening compositions include less than 5 wt. %, less than 4 wt. %, less than 3 wt. %, less than 2 wt. %, less than 1 wt. %, less than 0.5 wt. %, less than 0.1 wt. % of odium silicate, magnesium silicate, calcium silicate, and/or aluminum silicate.

Nonlimiting examples of mineral clays include montmorilonite, hectorite, saponite, bidelite, kaolinite, montmorillonites, saponites, laponites, hectorites (including disteardimonium hectorite), smectites, illites, vermiculite, mica, synthetic mica, and combinations thereof.

In various preferred embodiments, the mineral clay is laponite.

The total amount of the one or more mineral clays in the skin perfecting and tightening compositions will vary. Nonetheless, in various embodiments, the skin perfecting and tightening composition includes about 0.1 to about 10 wt. % of the one or more mineral clays, based on a total weight of the skin perfecting and tightening composition. In further embodiments, the skin perfecting and tightening composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, or about 2 to about 5 wt. % of the one or more mineral clays, based on a total weight of the skin perfecting and tightening composition.

### (d) Particulate Fillers

As the name suggests, particulate fillers are materials that exist as particulates in the skin perfecting and tightening compositions. The particulate fillers are generally essentially insoluble in water. The term “particulate” is construed broadly to include, for example, solid, hollow, or porous spherical or microspherical, flake, platelet, irregular shaped particulates, but preferably will be a powder to assure uniform dispersal and homogeneous mechanical properties.

Nonlimiting examples of particulate fillers include includes metal oxides, metal hydroxides, metal carbonates, metal sulfates, various kinds of clay (other than the mineral clays of (c)), silica, alumina, powdered metals, glass microspheres, calcium carbonate, barium sulfate, sodium carbonate, magnesium carbonate, magnesium sulfate, barium carbonate, kaolin, carbon, calcium oxide, magnesium oxide, aluminum hydroxide, titanium dioxide, talc, mica, wollastonite latex particles, particles of thermoplastic elastomers, pulp powders, wood powders, cellulose derivatives, chitin, microbial cellulose, powders of crystalline, high melting polymers, beads of crosslinked polymers, organosilicone powders, and powders of polyacrylic acid, and the like, as well as combinations and derivatives thereof. The particulate fillers may optionally be surface modified with a hydrophobic modification.

Particulate fillers include waxes such as beeswax, montan wax, carnauba wax, candelilla wax, sugar cane wax, Japan wax, ozocerite, microcrystalline waxes, paraffin wax, lanolin wax, hydrogenated lanolin wax or acetylated lanolin wax.

Particulate fillers include soft focus powers. Soft focus powders are materials providing a blurring effect, typically due to their light-scattering properties on the skin. Such powders typically have high diffuse reflectance, low specular reflectance, and high diffuse transmittance. Soft focus powders give the skin a smoother appearance, for example, by reducing the difference in luminosity between the valley and the edges of wrinkles and imperfections.

Non-limiting examples of soft focus powders include powders of natural or synthetic origin such as mica, titanated mica, alumina, titanium dioxide, serecite, composite talc/titanium dioxide/alumina/silica powders, polyamide, poly(methyl (meth)acrylate), polyethylene powder, polymethylsilsesquioxane powder, sodium acrylates crosspolymer-2 and a mixture thereof. Additional non-limiting examples include calcium aluminum borosilicate (LUXSIL), PMMA (Microsphere M-100), polyethylene (POLYETHYLENE CI 2080), methyl methacrylate crosspolymer (COVABEADS LH85), nylon-12 (ORGASOL 2002), or ethylene/acrylic acid copolymer (FLOBEADS EA209).

In various embodiments, the skin perfecting and tightening compositions include at least one soft focus powder selected from silica (which may or may not be coated), fumed silica, silica silylate, composite talc/titanium dioxide/alumina/silica powders, polyamide (nylon), poly(methyl (meth)acrylate), polyethylene powder, polymethylsilsesquioxane powder, waxes, such as copernicia cerifera (carnauba) wax, dimethicone/vinyl dimethicone crosspolymer, nylon-12, cellulose, polylactic acid, boron nitride, and a mixture thereof. The copernicia cerifera (carnauba) wax can be provided as a dispersion non-water and alcohol. The dimethicone/vinyl dimethicone crosspolymer can be provided as silicone dispersion (INCI: Dimethicone/vinyl dimethicone crosspolymer (and) C12-14 Pareth-12). In some instances, the soft focus powder is (or includes) sodium acrylates crosspolymer-2, which is commercially available as AQUAKEEP 10SH-NFC as sodium acrylates crosspolymer-2 (and) water (and) silica.

The total amount of the one or more particulate fillers in the skin perfecting and tightening compositions will vary. Nonetheless, in various embodiments, the skin perfecting and tightening composition includes about 0.1 to about 10 wt. % of the one or more particulate fillers, based on a total weight of the skin perfecting and tightening composition. In further embodiments, the skin perfecting and tightening composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, or about 2 to about 5 wt. %, of the one or more particulate fillers, based on a total weight of the skin perfecting and tightening composition.

### (e) Water Soluble Organic Solvents

The skin perfecting and tightening compositions optionally include or exclude (or are essentially free from) one or more water soluble organic solvents. In a preferred embodiment, the skin perfecting and tightening composition includes one or more water soluble organic solvents.

The term “water soluble organic solvent” is interchangeable with the terms “water soluble solvent” and “water-miscible solvent” and means a compound that is liquid at 25° C. and at atmospheric pressure (760 mmHg), and it has a solubility of at least 50% in water under these conditions. In some cases, the water-soluble solvents has a solubility of at least 60%, 70%, 80%, or 90%. Non-limiting examples of water-soluble solvents include, for example, organic solvents selected from glycerin, alcohols (for example, C₁₋₁₂, C₁₋₁₀, C₁₋₈, or C₁₋₄ alcohols), polyols (polyhydric alcohols), glycols, and a mixture thereof.

Non-limiting examples of water soluble organic solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, or a mixture thereof.

The amount of the one or more water soluble organic solvents in the skin perfecting and tightening compositions, if present, will vary. Nonetheless, in various embodiments, the skin perfecting and tightening composition includes about 0.1 to about 10 wt. % of the one or more water soluble organic solvents, based on a total weight of the skin perfecting and tightening composition. In further embodiments, the skin perfecting and tightening composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, or about 1 to about 5 wt. % of the one or more water soluble organic solvents, based on a total weight of the skin perfecting and tightening composition.

### (f) Water

The amount of water in the skin perfecting and tightening compositions will vary. Nonetheless, in various embodiments, the skin perfecting and tightening composition include about 50 to about 90 wt. % water, based on a total weight of the skin perfecting and tightening composition. In further embodiments, the skin perfecting and tightening composition includes about 55 to about 90 wt. %, about 60 to about 90 wt. %, about 65 to about 90 wt. %, about 70 to about 90 wt. %, about 75 to about 90 wt. %, about 80 to about 90 wt. %, about 55 to about 85 wt. %, about 60 to about 85 wt. %, about 65 to about 85 wt. %, or about 70 to about 85 wt. % water, based on a total weight of the skin perfecting and tightening composition.

### (g) Fibers or Filaments

The skin perfecting compositions may optionally include one or more fibers or filaments. The term “fiber” is interchangeable with the term “filament” and refers to a slender threadlike material, as understood in the art. The fibers or filaments can be microfibers or nanofibers. Microfibers typically have a diameter of less than about 20, 15, or 10 micrometers. Nanofibers typically have a diameter of about 1 nm to about 1 μm. The use of fibers in the skin perfecting compositions improves the strength while maintaining or enhancing flexibility. The fibers are typically not appreciably soluble in the skin perfecting compositions, i.e., they retain their structure as a fiber or filament within the skin perfecting composition.

Nonlimiting examples of fibers include non-woven fibers, wood pulp fibers, cotton fibers, collagen fibers, modified collagen fibers, chitin fibers, modified chitin fibers, chitosan fibers, modified chitosan fibers, polysaccharide fibers, modified polysaccharide fibers, protein fibers, hydrolyzed protein fibers, fibrin fibers, silk protein fibers, silk fibrin fibers, peptide fibers, cellulose fibers, modified cellulose fibers, bio-cellulose fibers, acetic bio-cellulose fibers, bacterial cellulose fibers, chondroitin sulfate fibers, sodium alginate fivers, agar fibers, natural fibers, alginic acid fibers, cocoon filament and combination thereof.

In certain embodiments, the one or more fibers or filaments can be formed from a bioprotein derived from living organisms. Examples of bioproteins can include silk, cotton, porcine dermis, small intestine submucosa and bovine dermis or pericardium. In some embodiments, the fibers or filaments contemplated herein can be formed specifically of one or more animal-derived bioproteins. In other embodiments, the fibers or filaments may be formed more specifically of insect-derived bioproteins, for example, from spiders. In one particular embodiment, the bioprotein scaffolding can include a silk bioprotein, such as can be obtained, for example, from Bombyx mori silkworms. Other types of silk (and their silkworm sources) can include Muga (Antheraea assamensis silkworm), Eri (Sarnia cynthia ricini silkworm), or Pat (Bombyx textor silkworm).

As used herein, the term “bioprotein” includes any organic (i.e., not synthetic) material. When embodiments are described herein as being formed of a bioprotein material it is intended to mean formed primarily of organic material, and the inclusion of a portion of non-bioprotein material is contemplated. Thus, bioprotein products as disclosed herein include fibers or filaments that are at least 50% bioprotein, but may also be substantially (i.e., more than 90%, 95%, 99%, or even essentially 100%) bioprotein.

The total amount of the one or more fibers or filaments in the skin perfecting composition, if present, will vary. Nonetheless, in various embodiments, the skin perfecting composition includes about 0.01 to about 10 wt. % of the one or more fibers or filaments, based on a total weight of the skin perfecting composition. In further embodiments, the skin perfecting composition includes about 0.01 to about 8 wt. %, about 0.01 to about 6 wt. %, about 0.1 to about 10 wt. %, about 0.1 to about 8 wt. %, about 0.1 to about 5 wt. %, about 0.5 to about 10 wt. %, about 0.5 to about 8 wt. %, about 0.5 to about 6 wt. %, about 1 to about 10 wt. %, about 1 to about 8 wt. %, about 1 to about 6 wt. %, about 2 to about 10 wt. %, about 2 to about 8 wt. %, or about 2 to about 6 wt. %, based on a total weight of the skin perfecting composition.

### (h) Skin Active Agents

Nonlimiting examples of skin active agents include madecassoside, retinoic acid, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate; —derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia—extracts of flaxseed such as that sold under the name Linumine by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name Regu SEB by Pentapharm—mixtures of extracts of willowherb, of Terminalia chebula, nasturtium and of bioavailable zinc (microalgae), such as that sold under the name Seborilys Green Tech; —extracts of Pygeum africanum such as that sold under the name Pygeum africanum sterolic lipid extract by Euromed—extracts of Serenoa serrulata such as those sold under the name Viapure Sabal by Actives International, and those sold by the company Euromed—of extracts of plantain blends, Berberis aquifolium and sodium salicylate 20 such as that sold under the name Seboclear Rahn—extract of clove as that sold under the name Clove extract powder by Maruzen—argan oil such as that sold under the name Lipofructyl Laboratories Serobiologiques; 25—lactic protein filtrates, such as that sold under the name Normaseb by Sederma—the seaweed laminaria extracts, such as that sold under the name Laminarghane by Biotechmarine—oligosaccharides seaweed Laminaria digitata, such as that sold under the name Phycosaccharide 30 AC by the company Codif—extracts of sugar cane such as that sold under the name Policosanol by the company Sabinsa, the sulfonated shale oil, such as that sold under the name Ichtyol Pale by Ichthyol—extracts of meadowsweet (Spiraea ulmaria) such as that sold under the name Cytobiol Ulmaire by societeLibiol—sebacic acid, especially sold in the form of a sodium polyacrylate gel under the name Sebosoft by Sederma—glucomannans extracted from konjac tuber and modified with alkylsulfonate chains such as that sold under the name Biopol Beta by Arch Chemical—extracts of Sophora angustifolia, such as those sold under the name Sophora powder or Sophora extract by Bioland—extracts of cinchona bark succirubra such as that sold under the name Red Bark HS by Alban Muller—extracts of Quillaja saponaria such as that sold under the name 15 Panama wood HS by Alban Muller—glycine grafted onto an undecylenic chain, such as that sold under the name Lipacide UG OR by SEPPIC—the mixture of oleanolic acid and nordihydroguaiaretic acid, such as that sold under the form of a gel under the name AC.Net by Sederma; 20—phthalimidoperoxyhexanoic acid—citrate tri (C12-C13) sold under the name COSMACOL® ECI by Sasol; trialkyl citrate (C14-C15) sold under the name COSMACOL® ECL by Sasol—10-hydroxydecanoic acid, including mixtures acid-hydroxydecanoic October 25, sebacic acid and 1,10-decandiol such as that sold under the name Acnacidol BG by Vincience and mixtures thereof.

Skin active agents include cinnamic acid and derivatives thereof. Cinnamic acid or a derivative thereof may be selected from ferulic acid, p-coumaric acid, caffeic acid, sinapinic acid, chlorogenic acids, caftaric acid, chicoric acid, coutaric acid, rosmarinic acid, derivatives thereof, and combinations thereof. Equivalent derivatives thereof include those cinnamic acid derivatives having substitutions on the hydroxyl groups of the aromatic ring such as short chain aliphatic groups (one to six carbon atoms) or long chain aliphatic groups (seven to twenty-four carbon atoms) to form an ether, or such aliphatic groups substituted with alkyl, alkoxy, hydroxyl, amino, or amido, for example, to form a substituted ether. Equivalent derivatives thereof further include those cinnamic acid derivatives having modifications of the methoxy group(s) of the aromatic ring to short chain aliphatic groups (two to six carbon atoms) or to long chain aliphatic groups (seven to twenty-four carbon atoms) to form a longer chain ether, or such aliphatic groups substituted with alkyl, alkoxy, hydroxyl, amino, or amido, for example, to form a substituted long chain ether. The 3-carboxy group of a cinnamic acid derivative may also be converted to esters or amides having aliphatic groups of up to 24 carbons or an aromatic group, for example. Cis and trans isomers of the cinnamic acid derivatives are included herein since the cis isomer is readily converted to the trans isomer. Salts of the cinnamic acid derivatives are included herein. In one embodiment, the cinnamic acid derivative is a triethanolamine salt. Caffeic acid, also known as 3-(3,4-dihydroxyphenyl)-2-propenoic acid, is found in many fruits, vegetables, seasonings and beverages consumed by humans. Caffeic acid is present in such goods in conjugated forms such as chlorogenic acid. Para-coumaric acid, also known as 3-(4-hydroxyphenyl)-2-propenoic acid or p-hydroxycinnamic acid, is found in various plants, including lignin forming plants. Trans-ferulic acid, also known as 3-(4-hydroxy-3-methoxyphenyl)-2-propenoic acid or 4-hydroxy-3-methoxycinnamic acid, is also widely distributed in small amounts in plants. Sinapinic acid, also known as 3,5-dimethoxy4-hydroxycinnamic acid, is from black mustard seeds. Caffeic acid, para-coumaric acid, trans-ferulic acid and sinapinic acid are commercially available.

Skin active agents include hydroxy acids selected from alpha, beta or polyhydroxy acids. Nonlimiting examples include lactic acid, glycolic acid, salicylic acid, malic acid, tartaric acid, citric acid, mandelic acid, lactobionic acid, gluconolactone, galactose, and combinations thereof.

In various embodiments, one or more of the following skin active agents are selected from sodium hyaluronate, capryloyl salicylic acid, coco-caprylate/caprate, alpha and/or beta arbutin, ferulic acid, lucinol, kojic acid, resorcinol, tranexamic acid, gentisic acid, homogentisic, methyl gentisate or homogentisate, dioic acid, D pantheteine calcium sulphonate, lipoic acid, ellagic acid, vitamin B3, linoleic acid, stilbenoid, coumarin, tannin, curcuminoid, chalcone, phenylpropanoid, anthocyanin, dihydrochalcone, anthocyanidin, tocopherols, sesame lignin, vitamin C, caffeine, vitamin E, derivatives thereof, salts thereof, and a mixture thereof. In a preferred embodiment, the one or more skin agent agents are selected from Vitamin C, Vitamin E, caffeine, niacinamide, or a combination thereof.

Various skin active ingredients may be categorized as antioxidants, depigmenting agents, and anti-wrinkle agents.

### (i) Antioxidants

Examples of antioxidants include tocopherols (e.g. d-α-tocopherol, d-β-tocopherol, d-γ-tocopherol, d-delta-tocopherol), tocotrienols (e.g. d-α-tocotrienol, d-β-tocotrienol, d-γ-tocotrienol, d-delta-tocotrienol,) and vitamin E (α-tocopherol acetate). These compounds may be isolated from natural sources, prepared by synthetic means, or mixtures thereof. Tocotrienol-enriched vitamin E preparations may be obtained by fractionating vitamin E preparations to remove a portion of tocopherols and recover a preparation more highly concentrated in tocotrienol. Useful tocotrienols are natural products isolated, for example, from wheat germ oil, grain, or palm oil using high performance liquid chromatography, or isolated by alcohol extraction and/or molecular distillation from barley, brewer's grain or oats. As used herein, the term “tocotrienols” includes tocotrienol-rich-fractions obtained from these natural products as well as the pure compounds. The increased glutathione peroxidase activity protects the skin from oxidative damage.

Vitamin C and derivatives may be used, including ascorbic acid, sodium ascorbate, and the fat soluble esters tetrahexyldecyl ascorbate and ascorbyl palmitate, magnesium ascorbyl phosphate, ascorbyl-glucoside, glucosamine ascorbate, ascorbyl acetate, etc. Additionally, extracts from plants containing a high amount of vitamin C such as camu berry (Myrciaria dubia), acerola, Emblica officinalis, and bioflavonoids from rose hip and citrus may be used including water soluble bioflavonoids such as hesperidin methyl chalcone may also be used.

Sesame (Sesamum indicum) or sesame lignan may also be added. Sesame and its lignans (the fibrous compounds associated with the sesame) act as antioxidants. Sesame seed lignans significantly enhance vitamin E activity.

In addition, carotenoids, particularly the xanthophyll type, are also useful antioxidants that can be used. The xanthopyll type carotenoids include molecules, such as lutein, canthaxantin, cryptoxanthin, zeaxanthin and astaxanthin. Xanthophylls protect compounds, such as vitamin A, vitamin E, and other carotenoids.

Flavonoids can also function as antioxidants. In some instances, the flavonoid is a flavanone (derivative of 2,3-dihydro-2-phenylchromen-4-one). Flavones include: Butin, Eriodictyol, Hesperetin, Hesperidin, Homoeriodictyol, Isosakuranetin, Naringenin, Naringin, Pinocembrin, Poncirin, Sakuranetin, Sakuranin, and Sterubin. The flavonoid may be a flavanonol (derivative of 3-hydroxy-2,3-dihydro-2-phenylchromen-4-one). Flavanols include: Taxifolin, Aromadedrin, Chrysandroside A, Chrysandroside B, Xeractinol, Astilbin, and Fustin. The flavonoid may be a flavone (derivative of 2-phenylchromen-4-one). Flavones include: Apigenin, Luteolin, Tangeritin, Chrysin, Baicalein, Scutellarein, Wogonin, Synthetic Flavones: Diosmin, and Flavoxate. The flavonoid may be a flavonol (derivative of 3-hydroxy-2-phenylchromen-4-one). Flavonols include: 3-Hydroxyflavone, Azaleatin, Fisetin, Galangin, Gossypetin, Kaempferide, Kaempferol, Isorhamnetin, Morin, Myricetin, Natsudaidain, Pachypodol, Quercetin, Rhamnazin, Rhamnetin, Azalein, Hyperoside, Isoquercitin, Kaempferitrin, Myricitrin, Quercitrin, Robinin, Rutin, Spiraeoside, Xanthorhamnin, Amurensin, Icariin, and Troxerutin. The flavonoid may be a flavan-3-ol (derivatives of 2-phenyl-3,4-dihydro-2H-chromen-3-ol). Flavan-3-ols include:

Catechin, Epicatechin, Epigallocatechin, Epicatechin gallate, Epigallocatechin gallate, Epiafzelechin, Fisetinidol, Guibourtinidol, Mesquitol, and Robinetinidol. The flavonoid may be a flavan-4-ol (derivative of 2-phenylchroman-4-ol). Flavan-4-ols include: Apiforol and Luteoforol. The flavonoid may be an isoflavone (derivative of 3-phenylchromen-4-one). Isoflavones include: Genistein, Daidzein, Biochanin A, Formononetin, and the Equol metabolite from Daidzein.

The antioxidant may be an anthocyanidin (derivative of 2-phenylchromenylium cation). Anthocyanidins include: Aurantinidin, Cyanidin, Delphinidin, Europinidin, Luteolinidin, Pelargonidin, Malvidin, Peonidin, Petunidin, Rosinidin, and Xanthone.

The antioxidant may be a Dihydrochalcone (derivative of 1,3-diphenyl-1-propanone). Dihydrochalcones include: Phloretin, Dihydrochalcone phloretin Phlorizin, Aspalathin, Naringin dihydrochalcone, Neohesperidin dihydrochalcone, and Nothofagin. Without limiting the mode of action of the invention, dihydrochalcones may exert an antioxidant effect by reducing reactive free radicals, like reactive oxygen and reactive nitrogen species.

The antioxidant may be an anthocyanin. Anthocyanins and their derivatives are antioxidants. Anthocyanins encompasses a class of flavonoid compounds that are naturally occurring, water-soluble compounds, responsible for the red, purple, and blue colors of many fruits, vegetables, cereal grains, and flowers. Additionally, anthocyanins are collagenase inhibitors. The inhibition of collagenase helps in the prevention and reduction of wrinkles, increase in skin elasticity, etc., which are caused by a reduction in skin collagen. The anthocyanins may be obtained from any portion of various plant sources, such as the fruit, flower, stem, leaves, root, bark, or seeds. One of skill in the art will understand that certain portions of the plant may contain higher natural levels of anthocyanins, and, therefore, those portions are used to obtain the desired anthocyanins. In some instances, antioxidants may include one or more betacyanin. Betacyanins, like anthocyanins, may be obtained from natural sources and are antioxidants.

The antioxidant may be a Phenylpropanoid (derivatives of cinnamic acid). Phenylpropanoids include: Cinnamic acid, Caffeic acid, Ferulic acid, Trans-ferulic acid (including its antioxidant pharmacore 2,6-dihydroxyacetophenome), 5-Hydroxyferulic acid, Sinapic acid, Coumaryl alcohol, Coniferyl alcohol, Sinapyl alcohol, Eugenol, Chavicol, Safrole, P-coumaric acid, and Sinapinic acid. Without limiting the mode of action of the invention, Phenylpropanoids may neutralize free radicals.

The antioxidant may be a Chalcone (derivative of 1,3-diphenyl-2-propen-1-one). Chalcones include: Butein, Okanin, Carthamin, Marein, Sophoradin, Xanthohumol, Flavokvain A, Flavokavain B, Flavokavin C, and synthetic Safalcone.

The antioxidant may be a Curcuminoid. Curcuminoids include: Curcumin, Desmethoxycurcumin, bis-Desmethoxycurcumin, Tetrahydrocurcumin, and Tetrahydrocurcuminoids. Curcumin and tetrahydrocurcuminoids may be derived from rhizomes of Curcuma longa. Tetrahydrocurcumin, a metabolite of curcumin, has been found to be a more potent antioxidant and more stable compared to curcumin.

The antioxidant may be a Tannin. Tannins include: Tannin, Terflavin B, Glucogallin, Dgallic acid, and Quercitannic acid.

The antioxidant may be a stilbenoid. Stilbenoids include: Resveratrol,

Pterostilbene, and Piceatannol. Resveratrol may include, but is not limited to, 3,5,4′-trihydroxystilbene, 3,4,3′,5′-tetrahydroxystilbene (piceatannol), 2,3′,4,5′-tetrahydroxystilbene (oxyresveratrol), 4,4′-dihydroxystilbene, and alpha and beta glucoside, galactoside and mannoside derivatives thereof.

The antioxidant may be a Coumarin (derivatives of 2H-chromen-2-one). Coumarins include: 4-Hydroxycoumarin, Umbelliferone, Aesculetin, Herniarin,

### Auraptene, and Dicoumarol.

The antioxidant may be a Carotenoid. Carotenoids include: beta-Carotene, alpha-Carotene, gamma-Carotene, beta-Cryptoxanthin, Lycopene, Lutein, and Idebenone. Sesame (Sesamum indicum) or sesame lignan may also be added. Sesame and its lignans (the fibrous compounds associated with the sesame) act as antioxidants. Sesame seed lignans significantly enhance vitamin E activity.

The antioxidant may be: a Xanthone, Butylated Hydroxytoluene, 2,6-Di-tert-butylphenol, 2,4-Dimethyl-6-tert-butylphenol, Gallic acid, Eugenol, Uric acid, alpha-Lipoic acid, Ellagic acid, Chicoric acid, Chlorogenic acid, Rosmarinic acid, Salicylic acid, Acetylcysteine, S-Allyl cysteine, Barbigerone, Chebulagic acid, Edaravone, Ethoxyquin, Glutathione, Hydroxytyrosol, Idebenone, Melatonin, N-Acetylserotonin, Nordihydroguaiaretic acid, Oleocanthal, Oleuropein, Paradol, Piceatannol, Probucol, Propyl gallate, Protocatechuic acid, Pyritinol, Rutin, Secoisolariciresinol diglucoside, Sesamin, Sesamol, Silibinin, Silymarin, Theaflavin, Theaflavin digallate, Thmoquinone, Trolox, Tyrosol, Polyunsaturated fatty acids, and sulfur-based antioxidants such as Methionine or Lipoic acid.

### (ii) Depigmenting Agents

Nonlimiting examples of depigmenting agents include alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, certain compounds derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

(iii) Anti-Wrinkle Agent

The term “anti-wrinkle agent” refers to a natural or synthetic compound producing a biological effect, such as the increased synthesis and/or activity of certain enzymes, when brought into contact with an area of wrinkled skin, this has the effect of reducing the appearance of wrinkles and/or fine lines. Nonlimiting examples of anti-wrinkle agents include: desquamating agents, anti-glycation agents, inhibitors of NO-synthase, agents stimulating the synthesis of dermal or epidermal macromolecules and/or preventing their degradation, agents for stimulating the proliferation of fibroblasts and/or keratinocytes, or for stimulating keratinocyte differentiation reducing agents; muscle relaxants and/or dermo-decontracting agents, anti-free radical agents, and mixtures thereof. Examples of such compounds are: adenosine and its derivatives and retinoids other than retinol (as discussed above, such as retinol palmitate), ascorbic acid and its derivatives such as magnesium ascorbyl phosphate and ascorbyl glucoside; nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the α-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof. In at least one case, the skin perfecting and tightening composition includes adenosine derivatives, such as non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside. Other derivatives include adenosine receptor agonists such as adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′-N-ethylcarboxamido adenosine (CGS-21680), N-ethylcarboxamido-adenosine (NECA), the 5′ (N-cyclopropyl)-carboxamidoadenosine, DPMA (PD 129.944) and metrifudil.

The total amount of the one or more skin active agents in the skin perfecting and tightening compositions if present will vary. Nonetheless, in various embodiments, the skin perfecting and tightening compositions includes an amount greater than zero to about 10 wt. %, based on a total weight of the skin perfecting and tightening composition. In further embodiments, the skin perfecting and tightening composition includes an amount of skin active agents in an amount greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.1 to about 10 wt. %, about 0.1 to about 5 wt. %, about 0.1 to about 2.5 wt. %, about 0.1 to about 1 wt. %, about 0.1 to about 0.5 wt. %; about 1 to about 10 wt. %, about 1 to about 8 wt. %, about 1 to about 6 wt. %, about 1 to about 5 wt. %, about 1 to about 4 wt. %, about 1 to about 3 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %; about 4 to about 10 wt. %, about 4 to about 8 wt. %, or about 4 to about 6 wt. %, based on the total weight of the skin perfecting and tightening composition.

### (i) Miscellaneous Ingredients

The skin perfecting and tightening compositions of the instant disclosure may optionally include one or more miscellaneous ingredients. Miscellaneous ingredients are ingredients that are compatible with the skin perfecting and tightening compositions and do not disrupt or materially affect the basic and novel properties of the skin perfecting and tightening compositions. Miscellaneous ingredients commonly used in cosmetics are known in the art. Non-limiting examples include preservatives, fragrances, pH adjusters, salts, buffers, flavonoids, vitamins, botanical extracts, UV filtering agents, proteins, protein hydrolysates and/or isolates, hydrotropes, pearlescent agents, fillers, colorants, mattifying agents, etc.

In addition to the above, components described as optional component throughout the disclosure may also be construed as miscellaneous ingredients if not expressly set forth as an independent component of the skin perfecting and tightening composition. Thus, the term “miscellaneous ingredient” is understood as a catch-all phrase representing additional components that may optionally be present in amounts designated for the miscellaneous ingredients.

Miscellaneous ingredients can be included in the cosmetic composition, for example, in an amount of about 0.01 to about 10 wt. %, based on the total weight of the cosmetic composition. The total amount of the one or more miscellaneous ingredients may be about 0.01 to about 8 wt. %, about 0.01 to about 5 wt. %, about 0.01 to about 3 wt. %, about 0.1 to about 10 wt. %, about 0.1 to about 8 wt. %, about 0.1 to about 5 wt. %, about 0.1 to about 3 wt. %, about 1 to about 10 wt. %, about 1 to about 8 wt. %, about 1 to about 5 wt. %, or about 1 to about 3 wt. %, based on the total weight of the skin perfecting and tightening composition.

### (j) Organic Colorants

The skin perfecting and tightening compositions may optionally include one or more organic colorants. Non-limiting examples include D & C red no. 19 (CI 45,170), D & C red no. 9 (CI 15,585), D & C red no. 21 (CI 45,380), D & C orange no. 4 (CI 15,510), D & C orange no. 5 (CI 45,370), D & C red no. 27 (CI 45,410), D & C red no. 13 (CI 15,630), D & C red no. 7 (CI 15,850:1), D & C red no. 6 (CI 15,850:2), D & C yellow no. 5 (CI 19,140), D & C red no. 36 (CI 12,085), D & C orange no. 10 (CI 45,425), D & C yellow no. 6 (CI 15,985), D & C red no. 30 (CI 73,360), D & C red no. 3 (CI 45,430), carbon black (CI 77,266), cochineal carmine lake (CI 75,470), natural or synthetic melanin, and aluminium lakes.

The total amount of organic colorants, if present, may vary but is typically about 0.01 to about 20 wt. %, based on the total weigh of the skin perfecting and tightening composition. The total amount of organic colorants may be about 0.01 to about 20 wt. %, about 0.01 to about 18 wt. %, about 0.01 to about 16 wt. %, about 0.01 to about 14 wt. %, about 0.01 to about 12 wt. %, about 0.01 to about 10 wt. %, about 0.01 to about 8 wt. %, about 0.01 to about 6 wt. %, about 0.01 to about 5 wt. %, about 0.01 to about 4 wt. %, about 0.1 to about 10 wt. %, about 0.1 to about 8 wt. %, about 0.1 to about 5 wt. %, or about 0.1 to about 4 wt. %, about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %, about 2 to about 20 wt. %, about 2 to about 18 wt. %, about 2 to about 16 wt. %, about 2 to about 14 wt. %, about 2 to about 12 wt. %, about 2 to about 10 wt. %, about 2 to about 8 wt. %, about 4 to about 20 wt. %, about 4 to about 18 wt. %, about 4 to about 16 wt. %, about 4 to about 14 wt. %, about 4 to about 12 wt. %, about 4 to about 10 wt. %, about 4 to about 8 wt. %, including ranges and subranges thereof, based on the total weight of the skin perfecting and tightening composition.

### EMBODIMENTS

In various embodiments the skin perfecting and tightening composition comprises or consists of:
(a) about 1 to about 15 wt. %, preferably about 1 to about 10 wt. %, more preferably about 1 to about 5 wt. % of pullulan;
(b) optionally, up to about 12 wt. %, preferably about 0.1 to about 10 wt. %, more preferably about 1 to about 5 wt. % of one or more hydrophilic non-acrylate film-forming polymers other than pullulan, preferably selected from one or more polyurethanes, more preferably selected from polyurethane-1, polyurethane-2, polyurethane-4, polyurethane-6, polyurethane-32, polyurethane-34, polyurethane-35, polyurethane-48, polyurethane-99, or a combination thereof;
(c) about 1 to about 10 wt. %, preferably about 1 to about 8 wt. %, more preferably about 2 to about 6 wt. % of one or more mineral clays, preferably one or more mineral clays selected from montmorilonite, hectorite, saponite, bidelite, kaolinite, montmorillonites, saponites, laponites, hectorites (including disteardimonium hectorite), smectites, illites, vermiculite, mica, synthetic mica, or a combination thereof, even more preferably at least one of the one or more mineral clays is laponite;
(d) about 1 to about 10 wt. %, preferably about 1 to about 8 wt. %, more preferably about 2 to about 6 wt. % of one or more particulate fillers, preferably wherein the one or more particulate fillers are selected from carnauba wax, fluorphlogopite, titanium dioxide, tin oxide, sodium acrylates crosspolymer-2, cellulose, perlite, silica, perlite, silica silylate, talc, mica, titanated mica, alumina, aluminum silicate, silica which may or may not be coated, fumed silica, polyamide, methyl methacrylate crosspolymer, poly(methyl (meth)acrylate), polyethylene powder, polymethylsilsesquioxane powder, nylon-12, cellulose, nylon-12, polyvinylalcohol crosspolymer, or a mixture thereof, more preferably wherein at least one of the one or more particulate fillers is silica;
(e) optionally, up to about 15 wt. %, preferably about 0.1 to about 12 wt. %, more preferably about 1 to about 10 wt. % of one or more water-soluble organic solvents, wherein the one or more water-soluble solvents are preferably selected from glycerin, mono-alcohols, polyols (polyhydric alcohols), glycols, and a mixture thereof, for example, one or more of ethyl alcohol, isopropyl alcohol, propyl alcohol, benzyl alcohol, phenylethyl alcohol, of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, or a mixture thereof;
(f) about 60 to about 92 wt. %, preferably about 70 to about 90 wt. %, more preferably about 75 to about 90 wt. % water;
   wherein all weight percentages are based on a total weight of the composition.

The skin perfecting and tightening composition may optionally further one or more additional component. Nonlimiting examples include (g) one or more fibers, (h) one or more skin active agents, (i) one or more miscellaneous ingredients.

In various embodiments, the skin perfecting and tightening composition are optionally free or essentially free from one or more components. Nonlimiting examples include volatile hydrocarbons (e.g., isoparaffins, isohexadecane, isododecane, isodecane, undecane, tridecane, dodecane, isohexyl, isodecyl, neopentanoate, and a combination thereof, etc.), styrene/butadiene copolymer, silica silylate, silicones, dimethicone crosspolymers, C₁-C₄ alkyl(methacrylate) polymers, acrylates/isobornyl acrylate copolymer, sodium polyacrylate, and/or acrylate and/or methacrylate polymers and copolymers. In a preferred embodiment, the skin perfecting and tightening compositions are free from acrylate and/or methacrylate polymers and copolymers (e.g., sodium polyacrylate, acrylates copolymer, acrylates/isobornyl acrylate copolymer, C₁-C₄ alkyl(methacrylate) polymers) regardless of what other monomer units appear in the polymer and copolymer (and crosspolymer).

In various embodiments the skin perfecting and tightening composition comprises or consists of:
(a) about 1 to about 15 wt. %, preferably about 1 to about 10 wt. %, more preferably about 1 to about 5 wt. % of pullulan;
(b) optionally, up to about 12 wt. %, preferably about 0.1 to about 10 wt. %, more preferably about 1 to about 5 wt. % of one or more hydrophilic non-acrylate film-forming polymers other than pullulan, preferably selected from one or more polyurethanes, more preferably selected from polyurethane-1, polyurethane-2, polyurethane-4, polyurethane-6, polyurethane-32, polyurethane-34, polyurethane-35, polyurethane-48, polyurethane-99, even more preferably polyurethane-99;
(c) about 1 to about 10 wt. %, preferably about 1 to about 8 wt. %, more preferably about 2 to about 6 wt. % of one or more mineral clays, preferably one or more mineral clays selected from montmorilonite, hectorite, saponite, bidelite, kaolinite, montmorillonites, saponites, laponites, hectorites (including disteardimonium hectorite), smectites, illites, vermiculite, mica, synthetic mica, or a combination thereof, even more preferably at least one of the one or more mineral clays is laponite;
(d) about 1 to about 10 wt. %, preferably about 1 to about 8 wt. %, more preferably about 2 to about 6 wt. % of one or more particulate fillers, preferably wherein the one or more particulate fillers are selected from carnauba wax, fluorphlogopite, titanium dioxide, tin oxide, sodium acrylates crosspolymer-2, cellulose, perlite, silica, perlite, silica silylate, talc, mica, titanated mica, alumina, aluminum silicate, silica which may or may not be coated, fumed silica, polyamide, methyl methacrylate crosspolymer, poly(methyl (meth)acrylate), polyethylene powder, polymethylsilsesquioxane powder, nylon-12, cellulose, nylon-12, polyvinylalcohol crosspolymer, or a mixture thereof, more preferably wherein at least one of the one or more particulate fillers is silica;
(e) optionally, up to about 15 wt. %, preferably about 0.1 to about 12 wt. %, more preferably about 1 to about 10 wt. % of one or more water-soluble organic solvents, wherein the one or more water-soluble solvents are preferably selected from glycerin, mono-alcohols, polyols (polyhydric alcohols), glycols, and a mixture thereof, for example, one or more of ethyl alcohol, isopropyl alcohol, propyl alcohol, benzyl alcohol, phenylethyl alcohol, of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, or a mixture thereof;
(f) about 60 to about 92 wt. %, preferably about 70 to about 90 wt. %, more preferably about 75 to about 90 wt. % water;
(g) optionally, up to about 10 wt. %, preferably about 0.1 to about 8 wt. %, more preferably about 1 to about 6 wt. % of one or more fibers, wherein the one or more fibers are selected from non-woven fibers, wood pulp fibers, cotton fibers, collagen fibers, modified collagen fibers, chitin fibers, modified chitin fibers, chitosan fibers, modified chitosan fibers, polysaccharide fibers, modified polysaccharide fibers, protein fibers, hydrolyzed protein fibers, fibrin fibers, silk protein fibers, silk fibrin fibers, peptide fibers, cellulose fibers, modified cellulose fibers, bio-cellulose fibers, acetic bio-cellulose fibers, bacterial cellulose fibers, chondroitin sulfate fibers, sodium alginate fivers, agar fibers, natural fibers, alginic acid fibers, cocoon filament and combination thereof, more preferably wherein at least one of the one or more fibers is a protein fiber, a hydrolyzed protein fiber, a silk protein fiber, a fibrin fiber, a silk fibrin fiber, a spider silk fiber, or a combination thereof;
(h) optionally, up to about 10 wt. %, preferably about 0.01 to about 8 wt. %, more preferably about 0.1 to about 8 wt. % of one or more skin active agents, preferably wherein at least one of the one or more skin active agents is selected from antioxidants, anti-wrinkle agents, depigmenting agents, vitamins, ceramides, alpha and/or beta hydroxy acids, anti-acne agents, or a mixture thereof, for example, vitamin A, vitamin C, vitamin D, vitamin E, niacinamide, caffeine, ceramides, ferulic acid, salicylic acid, madecassoside, retinoic acid, benzoyl peroxide, or a combination thereof; and
(i) optionally, up to about 10 wt. %, preferably about 0.1 to about 10 wt. %, more preferably about 1 to about 8 wt. % of one or more miscellaneous ingredients, preferably wherein the one or more miscellaneous ingredients are selected from preservatives, fragrances, pH adjusters, salts, buffers, flavonoids, vitamins, botanical extracts, UV filtering agents, proteins, protein hydrolysates and/or isolates, hydrotropes, pearlescent agents, fillers, colorants, mattifying agents, or a combination thereof;
(j) optionally, one or more organic colorants, for example, about 0.01 to about 15 wt. %, preferably about 0.1 to about 10 wt. %, or about 0.1 to about 5 wt. % of one or more organic colorants;
   wherein all weight percentages are based on a total weight of the composition; and
   wherein the composition is optionally, free or essentially free from acrylate and/or methacrylate polymers and copolymers (e.g., sodium polyacrylate, acrylates copolymer, acrylates/isobornyl acrylate copolymer, C₁-C₄ alkyl(methacrylate) polymers) regardless of what other monomer units appear in the polymer and copolymer (and crosspolymer).

The skin perfecting and tightening composition may optionally be free or essentially free from one or more components. Nonlimiting examples include volatile hydrocarbons (e.g., isoparaffins, isohexadecane, isododecane, isodecane, undecane, tridecane, dodecane, isohexyl, isodecyl, neopentanoate, and a combination thereof, etc.), hydrogenated styrene/butadiene copolymer, silica silylate, silicones, dimethicone crosspolymers, C₁-C₄ alkyl(methacrylate) polymers, acrylates/isobornyl acrylate copolymer, sodium polyacrylate, and/or acrylate and/or methacrylate polymers and copolymers. In a preferred embodiment, the skin perfecting and tightening compositions are free from acrylate and/or methacrylate polymers and copolymers (e.g., sodium polyacrylate, acrylates copolymer, acrylates/isobornyl acrylate copolymer, C₁-C₄ alkyl(methacrylate) polymers) regardless of what other monomer units appear in the polymer and copolymer (and crosspolymer).

In various embodiments the skin perfecting and tightening composition comprises or consists of:
(a) about 1 to about 15 wt. %, preferably about 1 to about 10 wt. %, more preferably about 1 to about 5 wt. % of pullulan;
(b) optionally, up to about 12 wt. %, preferably about 0.1 to about 10 wt. %, more preferably about 1 to about 5 wt. % of one or more polyurethanes, preferably one or more polyurethanes selected from polyurethane-1, polyurethane-2, polyurethane-4, polyurethane-6, polyurethane-32, polyurethane-34, polyurethane-35, polyurethane-48, polyurethane-99, or a combination thereof, more preferably at least one of the one or more polyurethanes is polyurethane-99;
   wherein (a) and (b), if present, are in a weight ratio of (a) to (b) from about 1:5 to about 5:1, preferably about 1:3 to about 3:1, more preferably about 1:2 to about 2:1;
(c) about 1 to about 10 wt. %, preferably about 1 to about 8 wt. %, more preferably about 2 to about 6 wt. % of laponite;
(d) about 1 to about 10 wt. %, preferably about 1 to about 8 wt. %, more preferably about 2 to about 6 wt. % of one or more particulate fillers, preferably wherein the one or more particulate fillers are selected from carnauba wax, fluorphlogopite, titanium dioxide, tin oxide, sodium acrylates crosspolymer-2, cellulose, perlite, silica, perlite, silica silylate, talc, mica, titanated mica, alumina, aluminum silicate, silica which may or may not be coated, fumed silica, polyamide, methyl methacrylate crosspolymer, polyethylene powder, polymethylsilsesquioxane powder, nylon-12, cellulose, nylon-12, polyvinylalcohol crosspolymer, or a mixture thereof, more preferably wherein at least one of the one or more particulate fillers is a silica;
(e) optionally, up to about 15 wt. %, preferably about 0.1 to about 12 wt. %, more preferably about 1 to about 10 wt. % of one or more water-soluble organic solvents, wherein the one or more water-soluble solvents are preferably selected from glycerin, mono-alcohols, polyols (polyhydric alcohols), glycols, and a mixture thereof, for example, one or more of ethyl alcohol, isopropyl alcohol, propyl alcohol, benzyl alcohol, phenylethyl alcohol, of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, or a mixture thereof;
(f) about 60 to about 92 wt. %, preferably about 70 to about 90 wt. %, more preferably about 75 to about 90 wt. % water;
(g) optionally, up to about 10 wt. %, preferably about 0.1 to about 8 wt. %, more preferably about 1 to about 6 wt. % of one or more fibers, wherein the one or more fibers are selected from non-woven fibers, wood pulp fibers, cotton fibers, collagen fibers, modified collagen fibers, chitin fibers, modified chitin fibers, chitosan fibers, modified chitosan fibers, polysaccharide fibers, modified polysaccharide fibers, protein fibers, hydrolyzed protein fibers, fibrin fibers, silk protein fibers, silk fibrin fibers, peptide fibers, cellulose fibers, modified cellulose fibers, bio-cellulose fibers, acetic bio-cellulose fibers, bacterial cellulose fibers, chondroitin sulfate fibers, sodium alginate fivers, agar fibers, natural fibers, alginic acid fibers, cocoon filament and combination thereof, more preferably wherein at least one of the one or more fibers is a protein fiber, a hydrolyzed protein fiber, a silk protein fiber, a fibrin fiber, a silk fibrin fiber, a spider silk fiber, or a combination thereof;
(h) optionally, up to about 10 wt. %, preferably about 0.01 to about 8 wt. %, more preferably about 0.1 to about 8 wt. % of one or more skin active agents, preferably wherein at least one of the one or more skin active agents is selected from antioxidants, anti-wrinkle agents, depigmenting agents, vitamins, ceramides, alpha and/or beta hydroxy acids, anti-acne agents, or a mixture thereof, for example, vitamin A, vitamin C, vitamin D, vitamin E, niacinamide, caffeine, ceramides, ferulic acid, salicylic acid, madecassoside, retinoic acid, benzoyl peroxide, or a combination thereof; and
(i) optionally, up to about 10 wt. %, preferably about 0.1 to about 10 wt. %, more preferably about 1 to about 8 wt. % of one or more miscellaneous ingredients, preferably wherein the one or more miscellaneous ingredients are selected from preservatives, fragrances, pH adjusters, salts, buffers, flavonoids, vitamins, botanical extracts, UV filtering agents, proteins, protein hydrolysates and/or isolates, hydrotropes, pearlescent agents, fillers, colorants, mattifying agents, or a combination thereof;
(j) optionally, one or more organic colorants, for example, about 0.01 to about 15 wt. %, preferably about 0.1 to about 10 wt. %, or about 0.1 to about 5 wt. % of one or more organic colorants;
   wherein all weight percentages are based on a total weight of the composition; and
   wherein the composition is preferably free or essentially free from acrylate and/or methacrylate polymers and copolymers (e.g., sodium polyacrylate, acrylates copolymer, acrylates/isobornyl acrylate copolymer, C₁-C₄ alkyl(methacrylate) polymers) regardless of what other monomer units appear in the polymer and copolymer (and crosspolymer).

The skin perfecting and tightening composition may optionally be free or essentially free from one or more components. Nonlimiting examples include volatile hydrocarbons (e.g., isoparaffins, isohexadecane, isododecane, isodecane, undecane, tridecane, dodecane, isohexyl, isodecyl, neopentanoate, and a 5 combination thereof, etc.), hydrogenated styrene/butadiene copolymer, silica silylate, silicones, dimethicone crosspolymers, C₁-C₄ alkyl(methacrylate) polymers, acrylates/isobornyl acrylate copolymer, sodium polyacrylate, and/or acrylate and/or methacrylate polymers and copolymers.

In various embodiments the skin perfecting and tightening composition comprises or consists of:
(a) about 1 to about 15 wt. %, preferably about 1 to about 10 wt. %, more preferably about 1 to about 5 wt. % of pullulan;
(b) optionally, up to about 12 wt. %, preferably about 0.1 to about 10 wt. %, more preferably about 1 to about 5 wt. % of polyurethane-99;
   wherein (a) and (b), if present, are in a weight ratio of (a) to (b) from about 1:5 to about 5:1, preferably about 1:3 to about 3:1, more preferably about 1:2 to about 2:1;
(c) about 1 to about 10 wt. %, preferably about 1 to about 8 wt. %, more preferably about 2 to about 6 wt. % of laponite;
(d) about 1 to about 10 wt. %, preferably about 1 to about 8 wt. %, more preferably about 2 to about 6 wt. % of one or more particulate fillers, preferably wherein at least one of the one or more particulate fillers is a silica;
(e) optionally, up to about 15 wt. %, preferably about 0.1 to about 12 wt. %, more preferably about 1 to about 10 wt. % of one or more water-soluble organic solvents, wherein the one or more water-soluble solvents are preferably selected from glycerin, mono-alcohols, polyols (polyhydric alcohols), glycols, and a mixture thereof;
(f) about 60 to about 92 wt. %, preferably about 70 to about 90 wt. %, more preferably about 75 to about 90 wt. % water;
(g) optionally, up to about 10 wt. %, preferably about 0.1 to about 8 wt. %, more preferably about 1 to about 6 wt. % of one or more fibers, wherein the one or more fibers are preferably selected from a protein fiber, a hydrolyzed protein fiber, a silk protein fiber, a fibrin fiber, a silk fibrin fiber, a spider silk fiber, or a combination thereof;
(h) optionally, up to about 10 wt. %, preferably about 0.01 to about 8 wt. %, more preferably about 0.1 to about 8 wt. % of one or more skin active agents; and
(i) optionally, up to about 10 wt. %, preferably about 0.1 to about 10 wt. %, more preferably about 1 to about 8 wt. % of one or more miscellaneous ingredients, preferably wherein the one or more miscellaneous ingredients are selected from preservatives, fragrances, pH adjusters, salts, buffers, flavonoids, vitamins, botanical extracts, UV filtering agents, proteins, protein hydrolysates and/or isolates, hydrotropes, pearlescent agents, fillers, colorants, mattifying agents, or a combination thereof;
(j) optionally, one or more organic colorants, for example, about 0.01 to about 15 wt. %, preferably about 0.1 to about 10 wt. %, or about 0.1 to about 5 wt. % of one or more organic colorants;
   wherein all weight percentages are based on a total weight of the composition;
   wherein the composition is free or essentially free of silicones (e.g., silicone oils, silicone elastomers, and compounds containing a silicone atom);
   wherein the composition is preferably free or essentially free from acrylate and/or methacrylate polymers and copolymers (e.g., sodium polyacrylate, acrylates copolymer, acrylates/isobornyl acrylate copolymer, C₁-C₄ alkyl(methacrylate) polymers) regardless of what other monomer units appear in the polymer and copolymer (and crosspolymer).

The skin perfecting and tightening composition may optionally be free or essentially free from one or more components. Nonlimiting examples include volatile hydrocarbons (e.g., isoparaffins, isohexadecane, isododecane, isodecane, undecane, tridecane, dodecane, isohexyl, isodecyl, neopentanoate, and a combination thereof, etc.), hydrogenated styrene/butadiene copolymer, silica silylate, silicones, dimethicone crosspolymers, C₁-C₄ alkyl(methacrylate) polymers, acrylates/isobornyl acrylate copolymer, sodium polyacrylate, and/or acrylate and/or methacrylate polymers and copolymers.

pH

The pH of the skin perfecting and tightening compositions will vary. Nonetheless, in various embodiments, the skin perfecting and tightening composition and tightening has a pH of about 4 to about 9. In further embodiments, skin perfecting and tightening composition has a pH of about 4 to about 8, about 4 to about 7.5, about 5 to about 9, about 5 to about 8, about 5 to about 7.5, about 5.5 to about 9, about 5.5 to about 8, or about 5.5 to about 8.5.

### Optional Features

In various embodiments, the skin perfecting and tightening compositions described throughout the instant disclosure are free or essentially free from hydrogenated styrene/butadiene copolymer. In other embodiments, the skin perfecting and tightening composition includes less than 1 wt. % of hydrogenated styrene/butadiene copolymer. In further embodiments, the skin perfecting and tightening composition includes less than 0.8 wt. %, less than 0.5 wt. %, less than 0.1 wt. %, or less than 0.05 wt. % of hydrogenated styrene/butadiene copolymer. In other embodiments, the skin perfecting and tightening compositions optionally include up to about 2, 3, or 5 wt. % of hydrogenated styrene/butadiene copolymer.

In various embodiments, the skin perfecting and tightening compositions of the instant disclosure are free or essentially free from silica silylate. In other embodiments, the skin perfecting and tightening composition includes less than 1 wt. % of silica silylate. In further embodiments, the skin perfecting and tightening composition includes less than 0.8 wt. %, less than 0.5 wt. %, less than 0.1 wt. %, or less than 0.05 wt. % of silica silylate. In other embodiments, the skin perfecting and tightening compositions optionally include up to about 2, 3, or 5 wt. % of silica silylate.

In various embodiments, the skin perfecting and tightening compositions of the instant disclosure are free or essentially free from silicones. In other embodiments, the skin perfecting and tightening composition includes less than 1 wt. % of silicones. In further embodiments, the skin perfecting and tightening composition includes less than 0.8 wt. %, less than 0.5 wt. %, less than 0.1 wt. %, or less than 0.05 wt. % of silicones. In other embodiments, the skin perfecting and tightening compositions optionally include up to about 2, 3, or 5 wt. % of silicones.

In various embodiments, the skin perfecting and tightening compositions of the instant disclosure are free or essentially free from dimethicone crosspolymers. In other embodiments, the skin perfecting and tightening composition includes less than 1 wt. % of dimethicone crosspolymers. In further embodiments, the skin perfecting and tightening composition includes less than 0.8 wt. %, less than 0.5 wt. %, less than 0.1 wt. %, or less than 0.05 wt. % of dimethicone crosspolymers. In other embodiments, the skin perfecting and tightening compositions optionally include up to about 2, 3, or 5 wt. % of dimethicone crosspolymers.

In various embodiments, the skin perfecting and tightening compositions of the instant disclosure are free or essentially free from C₁-C₄ alkyl(methacrylate) polymer. In other embodiments, the skin perfecting and tightening composition includes less than 1 wt. % of C₁-C₄ alkyl(methacrylate) polymer. In further embodiments, the skin perfecting and tightening composition includes less than 0.8 wt. %, less than 0.5 wt. %, less than 0.1 wt. %, or less than 0.05 wt. % of C₁-C₄ alkyl(methacrylate) polymer. In other embodiments, the skin perfecting and tightening compositions optionally include up to about 2, 3, or 5 wt. % of C₁-C₄ alkyl(methacrylate) polymer.

In various embodiments, the skin perfecting and tightening compositions of the instant disclosure are free or essentially free from acrylates/isobornyl acrylate copolymer. In other embodiments, the skin perfecting and tightening composition includes less than 1 wt. % of acrylates/isobornyl acrylate copolymer. In further embodiments, the skin perfecting and tightening composition includes less than 0.8 wt. %, less than 0.5 wt. %, less than 0.1 wt. %, or less than 0.05 wt. % of acrylates/isobornyl acrylate copolymer. In other embodiments, the skin perfecting and tightening compositions optionally include up to about 2, 3, or 5 wt. % of acrylates/isobornyl acrylate copolymer.

In various embodiments, the skin perfecting and tightening compositions of the instant disclosure are free or essentially free from sodium polyacrylate. In other embodiments, the skin perfecting and tightening composition includes less than 1 wt. % of sodium polyacrylate. In further embodiments, the skin perfecting and tightening composition includes less than 0.8 wt. %, less than 0.5 wt. %, less than 0.1 wt. %, or less than 0.05 wt. % of sodium polyacrylate. In other embodiments, the skin perfecting and tightening compositions optionally include up to about 2, 3, or 5 wt. % of sodium polyacrylate.

In various embodiments, the skin perfecting and tightening compositions of the instant disclosure are free or essentially free from acrylate and/or methacrylate polymers and copolymers. In other embodiments, the skin perfecting and tightening composition includes less than 1 wt. % of acrylate and/or methacrylate polymers and copolymers. In further embodiments, the skin perfecting and tightening composition includes less than 0.8 wt. %, less than 0.5 wt. %, less than 0.1 wt. %, or less than 0.05 wt. % of acrylate and/or methacrylate polymers and copolymers. In other embodiments, the skin perfecting and tightening compositions optionally include up to about 2, 3, or 5 wt. % of acrylate and/or methacrylate polymers and copolymers.

In various embodiments, the skin perfecting and tightening compositions of the instant disclosure are free or essentially free from volatile hydrocarbons (e.g., isoparaffins, isohexadecane, isododecane, isodecane, undecane, tridecane, dodecane, isohexyl, isodecyl, neopentanoate, and a combination thereof, etc.). In other embodiments, the skin perfecting and tightening composition includes less than 1 wt. % of volatile hydrocarbons. In further embodiments, the skin perfecting and tightening composition includes less than 0.8 wt. %, less than 0.5 wt. %, less than 0.1 wt. %, or less than 0.05 wt. % of volatile hydrocarbons. In other embodiments, the skin perfecting and tightening compositions optionally include up to about 2, 3, or 5 wt. % of volatile hydrocarbons. Nonlimiting examples of volatile hydrocarbons that may optionally be included or excluded include isoparaffin, isohexadecane, isododecane, isodecane, undecane, tridecane, dodecane, isohexyl, isodecyl, neopentanoate, or a combination thereof.

### Methods

The skin perfecting and tightening compositions are particularly useful for improving the appearance of skin, especially the skin of a human. When the skin perfectings and tightening compositions are applied to the skin, they provide an immediate improvement to the appearance of the skin that is long lasting. The skin perfecting and tightening compositions are particularly useful in methods for:
reducing the appearance of fine lines of the skin;
reducing the appearance of wrinkles of the skin;
improving the tone of skin and/or improving the evenness of skin tone;
improving skin softness and/or smoothness;
reducing the appearance of eye bags;
reducing the appearance of dark circles around and/or below the eyes;
reducing the appearance of pores and/or scars; and/or
increasing the radiance, luminosity, and/or glow of the skin.

Typically, an effective amount of a skin perfecting and tightening composition is applied to the skin to be treated, for example, the skin of the face and/or neck. In some instances, it may be desirable to apply the skin perfecting and tightening composition to the skin around (or below) the eyes. The skin perfecting and tightening compositions can be applied with the hands or may be applies using a brush, sponge, tissue, cotton swab, fabric, or applicator (e.g., pen or other device), etc. The amount needed to achieve the desired effect can be ascertained by the consumer.

### EXAMPLES

Implementation of the present disclosure is provided by way of the following examples. The examples serve to illustrate the technology without being limiting in nature.

### Example 1

(Skin Perfecting Compositions)

| | | | 1 | 2 | 3 | 4 | 5 | 6 | 7 | 8 |
|---|---|---|---|---|---|---|---|---|---|---|
| | | | wt. % | | | | | | | |
| (a) | Pullulan | PULLULAN | 5 | 3.8 | 1.3 | | | | | |
| (b) | Hydrophilic | POLYURETHANE-99 | 3.8 | | 2.5 | 5 | 2.5 | 2.5 | | 5.0 |
| | Film | | | | | | | | | |
| | Former | | | | | | | | | |
| (c) | Mineral | LAPONITE | 3.8 | 3.4 | 3.8 | 3.4 | 2.5 | 2.9 | 3.8 | 3.4 |
| | Clay | | | | | | | | | |
| (d) | Particulate | SILICA | 4.3 | 3.5 | 3.5 | 2 | 2 | 2.0 | 2.8 | 4.3 |
| | Filler | | | | | | | | | |
| (e) | Solvent | GLYCERIN | 3 | 0.8 | 0.8 | 2.3 | 2.3 | 2.3 | 3.0 | 2.3 |

| | | | | | | | | | |
|---|---|---|---|---|---|---|---|---|---|
| (f) | Water | 80 | 87 | 88 | 87 | 91 | 90 | 90 | 85 |

The foregoing description illustrates and describes the disclosure. Additionally, the disclosure shows and describes only the preferred embodiments but as mentioned above, it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered a skin active agent and a miscellaneous ingredient. If a particular composition includes both a skin active agent and a miscellaneous ingredient, a single compound will serve as only the skin active agent or only the miscellaneous ingredient (the single compound does not serve as both the skin active agent and the miscellaneous ingredient).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The skin perfecting and tightening compositions of the instant disclosure may include one or more surfactants and/or emulsifiers, for example, one or more nonionic, anionic, cationic, and/or amphoteric/zwitterionic surfactants or emulsifiers. The terms “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, less than about 0.01 wt. % or none of the specified material.

The term “acrylate” is intended to include both acrylates and methacrylates and other alkylacrylates. The acrylate monomers used to make these polymers are normally alkyl acrylates of 4-17 carbon atoms, for example, 2-ethylhexyl acrylate, butyl acrylate, and isooctyl acrylate. Nonlimiting monomers of acrylate or methacrylate polymers and copolymers include acrylic acid, methacrylic acid, ethyl acrylate, methacrylate, and methyl methacrylate.

All components that are positively set forth in the instant disclosure may be negatively excluded from the claims, e.g., a claimed composition may be “free” or “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A skin perfecting and tightening composition comprising:
(a) about 1 to about 15 wt. % of pullulan;
(b) optionally, up to about 12 wt. % of one or more hydrophilic non-acrylate film-forming polymers other than pullulan, preferably one or more polyurethanes, more preferably, polyurethane-99;
(c) about 1 to about 10 wt. % of one or more mineral clays, preferably laponite;
(d) about 1 to about 10 wt. % of one or more particulate fillers; and
(e) optionally, one or more water-soluble organic solvents; and
(f) about 60 to about 90 wt. % water;
wherein all weight percentages are based on a total weight of the composition; and
wherein the composition is optionally free or essentially free from acrylate and/or methacrylate polymers and copolymers (e.g., sodium polyacrylate, acrylates copolymer, acrylates/isobornyl acrylate copolymer, C₁-C₄ alkyl(methacrylate) polymers) regardless of what other monomer units appear in the polymer and copolymer (and crosspolymer).

2. The composition of claim 1 comprising the one or more hydrophilic non-acrylate film-forming polymers and the one or more hydrophilic non-acrylate film-forming polymers are selected from polyurethanes, vinyl polymers, natural polymers, latex or pseudolatex polymers, vinylpyrrolidone-based polymers, amphoteric polymers, or a combination thereof, preferably one or more polyurethanes, more preferably, polyurethane-99.

3. The composition of claim 2, wherein the one or more hydrophilic non-acrylate film-forming polymers are selected polysaccharides and polysaccharide derivatives (e.g., carrageenan and dehydroxyxanthan gum), polyurethanes, polyvinyl alcohol, sodium hyaluronate, vinyl polymers, natural polymers, copolymers derived from C₄-C₈ monounsaturated carboxylic acids or anhydrides, methyl vinyl ether/butyl monomaleate copolymers, polyvinyl pyrollidone, polyvinyl acetate, water soluble gums, water soluble celluloses, dextrans, hyaluronic acid, cyclodextrins, polyvinyl caprolactam-polyvinyl acetate-polyethylene glycol graft copolymers, or a mixture thereof, preferably one or more polyurethanes, more preferably, polyurethane-99.

4. The composition of any one of the above claims, wherein the one or more mineral clays are selected from montmorilonite, hectorite, saponite, bidelite, kaolinite, montmorillonites, saponites, laponites, hectorites (including disteardimonium hectorite), smectites, illites, vermiculite, mica, synthetic mica, or a combination thereof, preferably wherein at least one of the one or more mineral clays is laponite.

5. The composition of any one of the above claims, wherein the one or more particulate fillers are selected from carnauba wax, fluorphlogopite, titanium dioxide, tin oxide, sodium acrylates crosspolymer-2, cellulose, perlite, silica, perlite, silica silylate, talc, mica, titanated mica, alumina, aluminum silicate, silica which may or may not be coated, fumed silica, polyamide, methyl methacrylate crosspolymer, poly(methyl (meth)acrylate), polyethylene powder, polymethylsilsesquioxane powder, nylon-12, cellulose, nylon-12, polyvinylalcohol crosspolymer, or a mixture thereof.

6. The composition of any one of the above claims, wherein the composition further comprises the one or more water-soluble organic solvents.

7. The composition of claim 6 comprising about 0.01 to about 15 wt. % of the one or more water-soluble organic solvents.

8. The composition of claim 6 or 7, wherein the one or more water-soluble organic solvents are selected from glycerin, mono-alcohols, polyols (polyhydric alcohols), glycols, and a mixture thereof.

9. The composition of any one of the above claims, further comprising:
(g) one or more fibers.

10. The composition of claim 8 comprising about 0.01 to about 15 wt. % of the one or more fibers.

11. The composition of claim 9, wherein the one or more fibers are selected from non-woven fibers, wood pulp fibers, cotton fibers, collagen fibers, modified collagen fibers, chitin fibers, modified chitin fibers, chitosan fibers, modified chitosan fibers, polysaccharide fibers, modified polysaccharide fibers, protein fibers, hydrolyzed protein fibers, fibrin fibers, silk protein fibers, silk fibrin fibers, peptide fibers, cellulose fibers, modified cellulose fibers, bio-cellulose fibers, acetic bio-cellulose fibers, bacterial cellulose fibers, chondroitin sulfate fibers, sodium alginate fivers, agar fibers, natural fibers, alginic acid fibers, cocoon filament and combination thereof.

12. The composition of any one of the above claims, further comprising:
(h) one or more skin active agents.

13. The composition of claim 12, wherein the one or more skin active agents are selected from vitamins (e.g., vitamin C, vitamin E), niacinamide, caffeine, or a combination thereof.

14. The composition of any one of the above claims, further comprising:
(i) one or more miscellaneous ingredients selected from preservatives, fragrances, pH adjusters, salts, buffers, flavonoids, vitamins, botanical extracts, UV filtering agents, proteins, protein hydrolysates and/or isolates, hydrotropes, pearlescent agents, fillers, colorants, mattifying agents, or a combination thereof.

15. The composition of any one of the above claims, wherein the composition is an emulsion, suspension, or dispersion.

16. The composition of any one of the above claims, wherein at least one of the one or more hydrophilic non-acrylate film-forming polymers is polyurethane-99.

17. The composition of any one of the above claims being free or essentially free from acrylate and methacrylate polymers and copolymers.

18. A skin perfecting and tightening composition comprising:
(a) about 1 to about 15 wt. % of pullulan;
(b) optionally, up to about 12 wt. % of one or more hydrophilic non-acrylate film-forming polymers, preferably one or more polyurethanes, more preferably, polyurethane-99;
(c) about 1 to about 10 wt. % of one or more mineral clays, preferably laponite;
(d) about 1 to about 10 wt. % of one or more particulate fillers;
(e) optionally, up to about 15 wt. % of one or more water-soluble organic solvents;
(f) about 60 to about 90 wt. % water;
(g) optionally, up to about 10 wt. % of one or more fibers;
(h) optionally, one or more skin active agents; and
(i) optionally, up to about 5 wt. % of one or more miscellaneous ingredients;
wherein all weight percentages are based on a total weight of the composition; and
wherein the composition is optionally free or essentially free from acrylate and/or methacrylate polymers and copolymers (e.g., sodium polyacrylate, acrylates copolymer, acrylates/isobornyl acrylate copolymer, C₁-C₄ alkyl(methacrylate) polymers) regardless of what other monomer units appear in the polymer and copolymer (and crosspolymer).

19. A method for treating skin comprising applying the skin perfecting and tightening composition of claim 1 to the skin.

20. The method of claim 19, wherein the skin perfecting and tightening composition is applied to the face of an individual using the individual's hands.


SEQUENCE LISTING (252 sequences)

SEQ ID NO: 1 - protein, 5 aa, synthetic construct
SYAMS

SEQ ID NO: 2 - protein, 5 aa, synthetic construct
SYAMS

SEQ ID NO: 3 - protein, 5 aa, synthetic construct
DYAMH

SEQ ID NO: 4 - protein, 5 aa, synthetic construct
YYEMS

SEQ ID NO: 5 - protein, 5 aa, synthetic construct
SYAMT

SEQ ID NO: 6 - protein, 17 aa, synthetic construct
AISGSGGSTYYADSVKG

SEQ ID NO: 7 - protein, 17 aa, synthetic construct
AISGSGGSTYYADSVKG

SEQ ID NO: 8 - protein, 17 aa, synthetic construct
GISWNSGSIGYADSVKG

SEQ ID NO: 9 - protein, 16 aa, synthetic construct
SIHYSGSTYYNPSLKS

SEQ ID NO: 10 - protein, 17 aa, synthetic construct
AISGSGDSTYYADSVKG

SEQ ID NO: 11 - protein, 9 aa, synthetic construct
VAMTGAFDI

SEQ ID NO: 12 - protein, 11 aa, synthetic construct
VVPAAYGAFDI

SEQ ID NO: 13 - protein, 9 aa, synthetic construct
VYQQGAFDI

SEQ ID NO: 14 - protein, 7 aa, synthetic construct
GQLRLDV

SEQ ID NO: 15 - protein, 11 aa, synthetic construct
ATLAVAGASDY

SEQ ID NO: 16 - protein, 12 aa, synthetic construct
RASQSVSSSYLA

SEQ ID NO: 17 - protein, 12 aa, synthetic construct
RASQSVSSSYLA

SEQ ID NO: 18 - protein, 11 aa, synthetic construct
RASQSISTYLN

SEQ ID NO: 19 - protein, 12 aa, synthetic construct
RASQSVSSSYLA

SEQ ID NO: 20 - protein, 7 aa, synthetic construct
GASSRAT

SEQ ID NO: 21 - protein, 7 aa, synthetic construct
GASSRAT

SEQ ID NO: 22 - protein, 7 aa, synthetic construct
AASSLQG

SEQ ID NO: 23 - protein, 7 aa, synthetic construct
GASSRAT

SEQ ID NO: 24 - protein, 10 aa, synthetic construct
QQYSSSPGFT

SEQ ID NO: 25 - protein, 10 aa, synthetic construct
QHYGNPPLYT

SEQ ID NO: 26 - protein, 9 aa, synthetic construct
QQSLMTPLT

SEQ ID NO: 27 - protein, 10 aa, synthetic construct
QHYGGSPLFT

SEQ ID NO: 28 - protein, 118 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARVAMTGAFDIWGKGTTVTVSS

SEQ ID NO: 29 - protein, 120 aa, synthetic construct
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCARVVPAAYGAFDIWGQGTMVTVSS

SEQ ID NO: 30 - protein, 118 aa, synthetic construct
EVQLVESGGGVVRPGGSLRLSCAASGFTFDDYAMHWVRQAPGKGLEWVSGISWNSGSIGYADSVKGRFTISRDNAKNSLYLQMNSLRAEDTALYYCVKVYQQGAFDIWGQGTTVTVSS

SEQ ID NO: 31 - protein, 115 aa, synthetic construct
QVQLVQSGGGLVQPGGSLRLSCAASSFDFDYYEMSWVRQAPGKGLEWIGSIHYSGSTYYNPSLKSRATISRDNSKNTLYLQMNTLRAEDTALYYCARGQLRLDVWGQGTTVTVSS

SEQ ID NO: 32 - protein, 120 aa, synthetic construct
EVQLVESGGGLVQPGGSLRLSCAASGFTFSSYAMTWVRQAPGKGLEWVSAISGSGDSTYYADSVKGRFTISRDNSKNTLNLQMNGLRSEDTAVYYCARATLAVAGASDYWGQGTTVTVSS

SEQ ID NO: 33 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPGFTFGPGTKVEIK

SEQ ID NO: 34 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFATYFCQHYGNPPLYTFGQGTKLEIK

SEQ ID NO: 35 - protein, 107 aa, synthetic construct
DIVMTQSPSSLSASVGDRITITCRASQSISTYLNWYQQKPGRAPKLLIHAASSLQGGVPSRFSGSGSGTYFTLTISSLQPEDFATYYCQQSLMTPLTFGGGTKVEIK

SEQ ID NO: 36 - protein, 109 aa, synthetic construct
EIVLTQSPGTLSLSPGERATLSCRASQSVSSSYLAWYQQKPGQAPRLLIYGASSRATGIPDRFGGSGSGTDFSLTISRLEPEDFAVYFCQHYGGSPLFTFGPGTKVDIK

SEQ ID NO: 37 - protein, 243 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARVAMTGAFDIWGKGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYSSSPGFTFGPGTKVEIKR

SEQ ID NO: 38 - protein, 245 aa, Homo sapiens
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCARVVPAAYGAFDIWGQGTMVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFATYFCQHYGNPPLYTFGQGTKLEIKR

SEQ ID NO: 39 - protein, 241 aa, Homo sapiens
EVQLVESGGGVVRPGGSLRLSCAASGFTFDDYAMHWVRQAPGKGLEWVSGISWNSGSIGYADSVKGRFTISRDNAKNSLYLQMNSLRAEDTALYYCVKVYQQGAFDIWGQGTTVTVSSGGGGSGGGGSGGGGSDIVMTQSPSSLSASVGDRITITCRASQSISTYLNWYQQKPGRAPKLLIHAASSLQGGVPSRFSGSGSGTYFTLTISSLQPEDFATYYCQQSLMTPLTFGGGTKVEIKR

SEQ ID NO: 40 - protein, 115 aa, Homo sapiens; identical to SEQ ID NO: 31

SEQ ID NO: 41 - protein, 245 aa, Homo sapiens
EVQLVESGGGLVQPGGSLRLSCAASGFTFSSYAMTWVRQAPGKGLEWVSAISGSGDSTYYADSVKGRFTISRDNSKNTLNLQMNGLRSEDTAVYYCARATLAVAGASDYWGQGTTVTVSSGGGGSGGGGSGGGGSEIVLTQSPGTLSLSPGERATLSCRASQSVSSSYLAWYQQKPGQAPRLLIYGASSRATGIPDRFGGSGSGTDFSLTISRLEPEDFAVYFCQHYGGSPLFTFGPGTKVDIKR

SEQ ID NO: 42 - protein, 21 aa, Homo sapiens
MALPVTALLLPLALLLHAARP

SEQ ID NO: 43 - protein, 20 aa, Homo sapiens
METDTLLLWVLLLWVPGSTG

SEQ ID NO: 44 - protein, 22 aa, Homo sapiens
MLLLVTSLLLCELPHPAFLLIP

SEQ ID NO: 45 - protein, 21 aa, Homo sapiens
MEFGLSWLFLVAILKGVQCSR

SEQ ID NO: 46 - protein, 15 aa, synthetic construct
GGGGSGGGGSGGGGS

SEQ ID NO: 47 - protein, 18 aa, synthetic construct
GSTSGSGKPGSGEGSTKG

SEQ ID NO: 48 - protein, 45 aa, Homo sapiens
TTTPAPRPPTPAPTIASQPLSLRPEACRPAAGGAVHTRGLDFACD

SEQ ID NO: 49 - protein, 39 aa, Homo sapiens
IEVMYPPPYLDNEKSNGTIIHVKGKHLCPSPLFPGPSKP

SEQ ID NO: 50 - protein, 42 aa, Homo sapiens
AAAIEVMYPPPYLDNEKSNGTIIHVKGKHLCPSPLFPGPSKP

SEQ ID NO: 51 - protein, 12 aa, Homo sapiens
ESKYGPPCPPCP

SEQ ID NO: 52 - protein, 12 aa, Homo sapiens
ESKYGPPCPSCP

SEQ ID NO: 53 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 24 aa, Homo sapiens
IYIWAPLAGTCGVLLLSLVITLYC

SEQ ID NO: 55 - protein, 27 aa, Homo sapiens
FWVLVVVGGVLACYSLLVTVAFIIFWV

SEQ ID NO: 56 - protein, 28 aa, Homo sapiens
MFWVLVVVGGVLACYSLLVTVAFIIFWV

SEQ ID NO: 57 - protein, 42 aa, Homo sapiens
KRGRKKLLYIFKQPFMRPVQTTQEEDGCSCRFPEEEEGGCEL

SEQ ID NO: 58 - protein, 41 aa, Homo sapiens
RSKRSRLLHSDYMNMTPRRPGPTRKHYQPYAPPRDFAAYRS

SEQ ID NO: 59 - protein, 41 aa, Homo sapiens
RSKRSRGGHSDYMNMTPRRPGPTRKHYQPYAPPRDFAAYRS

SEQ ID NO: 60 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 25 aa, Homo sapiens
TTTPAPRPPTPAPTIASQPLSLRPE

SEQ ID NO: 63 - protein, 44 aa, Homo sapiens
ACRPAAGGAVHTRGLDFACDIYIWAPLAGTCGVLLLSLVITLYC

SEQ ID NO: 64 - protein, 5 aa, synthetic construct
SYWIE

SEQ ID NO: 65 - protein, 5 aa, synthetic construct
DYVIS

SEQ ID NO: 66 - protein, 5 aa, synthetic construct
THWMH

SEQ ID NO: 67 - protein, 5 aa, synthetic construct
GYWMY

SEQ ID NO: 68 - protein, 17 aa, synthetic construct
EILPGSGSTSYNEKFKG

SEQ ID NO: 69 - protein, 17 aa, synthetic construct
EIYPGSGSSYYNEKFKG

SEQ ID NO: 70 - protein, 17 aa, synthetic construct
MINPSDGVTYYAQTFQG

SEQ ID NO: 71 - protein, 17 aa, synthetic construct
AISPGGGSTYYPDSVKG

SEQ ID NO: 72 - protein, 11 aa, synthetic construct
RGYGYDEGFDY

SEQ ID NO: 73 - protein, 8 aa, synthetic construct
PGDLGFAY

SEQ ID NO: 74 - protein, 9 aa, synthetic construct
EYYGEGFDY

SEQ ID NO: 75 - protein, 12 aa, synthetic construct
SLTATHTYEYDY

SEQ ID NO: 76 - protein, 10 aa, synthetic construct
ASQDINSYLS

SEQ ID NO: 77 - protein, 14 aa, synthetic construct
ASQSVDYDGDSYMN

SEQ ID NO: 78 - protein, 11 aa, synthetic construct
RASQGISNYLA

SEQ ID NO: 79 - protein, 7 aa, synthetic construct
RANRLVD

SEQ ID NO: 80 - protein, 7 aa, synthetic construct
AASNLES

SEQ ID NO: 81 - protein, 7 aa, synthetic construct
SASNLQS

SEQ ID NO: 82 - protein, 9 aa, synthetic construct
LQYDEFPPT

SEQ ID NO: 83 - protein, 9 aa, synthetic construct
QQSNKDPFT

SEQ ID NO: 84 - protein, 9 aa, synthetic construct
QQSYSTPLT

SEQ ID NO: 85 - protein, 120 aa, synthetic construct
QVQLQQSGAELMKPGASVKISCKATGYTFSSYWIEWVKQRPGHGLEWIGEILPGSGSTSYNEKFKGKATFTADTSSNTAYMQLSSLTSEDSAVYYCARRGYGYDEGFDYWGQGTTLTVSS

SEQ ID NO: 86 - protein, 117 aa, synthetic construct
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 87 - protein, 118 aa, synthetic construct
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 88 - protein, 121 aa, synthetic construct
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS

SEQ ID NO: 89 - protein, 108 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 90 - protein, 112 aa, synthetic construct
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 91 - protein, 108 aa, synthetic construct
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 92 - protein, 5 aa, synthetic construct
SYAIS

SEQ ID NO: 93 - protein, 5 aa, synthetic construct
DYYIQ

SEQ ID NO: 94 - protein, 5 aa, synthetic construct
SYYMH

SEQ ID NO: 95 - protein, 5 aa, synthetic construct
NHYMH

SEQ ID NO: 96 - protein, 17 aa, synthetic construct
IIDPSDGNTNYAQNFQG

SEQ ID NO: 97 - protein, 17 aa, synthetic construct
WINPNSGGTSYAQKFQG

SEQ ID NO: 98 - protein, 17 aa, synthetic construct
GFDPEDGETIYAQKFQG

SEQ ID NO: 99 - protein, 17 aa, synthetic construct
WMNPNSGNTGYAQKFQG

SEQ ID NO: 100 - protein, 13 aa, synthetic construct
ERAAAGYYYYMDV

SEQ ID NO: 101 - protein, 10 aa, synthetic construct
EGDYYYGMDA

SEQ ID NO: 102 - protein, 8 aa, synthetic construct
DQGWGMDV

SEQ ID NO: 103 - protein, 9 aa, synthetic construct
SESGSDLDY

SEQ ID NO: 104 - protein, 11 aa, synthetic construct
RASQSISSYLN

SEQ ID NO: 105 - protein, 16 aa, synthetic construct
RSSQSLLHSNGYNYLD

SEQ ID NO: 106 - protein, 11 aa, synthetic construct
RASQSISSYLN

SEQ ID NO: 107 - protein, 11 aa, synthetic construct
RASQTIGNYVN

SEQ ID NO: 108 - protein, 7 aa, synthetic construct
AASSLQS

SEQ ID NO: 109 - protein, 7 aa, synthetic construct
LGSNRAS

SEQ ID NO: 110 - protein, 7 aa, synthetic construct
AASSLQS

SEQ ID NO: 111 - protein, 7 aa, synthetic construct
GASNLHT

SEQ ID NO: 112 - protein, 9 aa, synthetic construct
QQSYSTPLT

SEQ ID NO: 113 - protein, 9 aa, synthetic construct
MQGLQTPHT

SEQ ID NO: 114 - protein, 9 aa, synthetic construct
QQTYSTPYT

SEQ ID NO: 115 - protein, 9 aa, synthetic construct
QQTYSAPLT

SEQ ID NO: 116 - protein, 122 aa, synthetic construct
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 117 - protein, 119 aa, synthetic construct
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 118 - protein, 117 aa, synthetic construct
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 119 - protein, 118 aa, synthetic construct
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 120 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 121 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 122 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 123 - protein, 108 aa, synthetic construct
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 124 - protein, 546 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 125 - protein, 520 aa, Nipah virus
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 126 - protein, 83 aa, Nipah virus
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 127 - protein, 437 aa, Nipah virus
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 128 - protein, 500 aa, Nipah virus
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 129 - protein, 417 aa, Nipah virus
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 130 - protein, 500 aa, Nipah virus
ILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 131 - protein, 524 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 132 - protein, 498 aa, Nipah virus
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 133 - protein, 415 aa, Nipah virus
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 134 - protein, 602 aa, Nipah virus
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 135 - protein, 597 aa, Nipah virus
MGKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 136 - protein, 592 aa, Nipah virus
MGNTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 137 - protein, 587 aa, Nipah virus
MGKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 138 - protein, 582 aa, Nipah virus
MGSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 139 - protein, 577 aa, Nipah virus
MGSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 140 - protein, 573 aa, Nipah virus
MGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 141 - protein, 569 aa, Nipah virus
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 142 - protein, 546 aa, Henipavirus hendraense
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 143 - protein, 604 aa, Henipavirus hendraense
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 144 - protein, 526 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 145 - protein, 526 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 146 - protein, 569 aa, Nipah virus
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 147 - protein, 498 aa, Nipah virus; identical to SEQ ID NO: 132

SEQ ID NO: 148 - protein, 557 aa, unidentified
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 149 - protein, 545 aa, unidentified
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 150 - protein, 662 aa, unidentified
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 151 - protein, 36 aa, synthetic construct
  VARIANT 1..36: note = This sequence may encompass 1-6 GGGGGS repeating units
GGGGGSGGGGGSGGGGGSGGGGGSGGGGGSGGGGGS

SEQ ID NO: 152 - protein, 602 aa, Nipah virus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 153 - protein, 622 aa, unidentified
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 154 - protein, 632 aa, unidentified
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 155 - protein, 625 aa, unidentified
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 156 - protein, 552 aa, Nipah virus
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 157 - protein, 555 aa, Henipavirus hendraense
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 158 - protein, 26 aa, synthetic construct
MVVILDKRCYCNLLILILMISECSVG

SEQ ID NO: 159 - protein, 6 aa, synthetic construct
GGGGGS

SEQ ID NO: 160 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 161 - protein, 5 aa, synthetic construct
GGGGS

SEQ ID NO: 162 - protein, 600 aa, Nipah virus
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 163 - protein, 595 aa, Nipah virus
KVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 164 - protein, 590 aa, Nipah virus
NTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 165 - protein, 585 aa, Nipah virus
KGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 166 - protein, 580 aa, Nipah virus
SKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 167 - protein, 575 aa, Nipah virus
SYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 168 - protein, 571 aa, Nipah virus
TMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 169 - protein, 568 aa, Nipah virus
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 170 - protein, 603 aa, Henipavirus hendraense
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 171 - protein, 568 aa, Nipah virus
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 172 - protein, 621 aa, unidentified
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 173 - protein, 631 aa, unidentified
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 174 - protein, 624 aa, unidentified
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 175 - protein, 530 aa, unidentified
DFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 176 - protein, 640 aa, unidentified
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 177 - protein, 520 aa, Henipavirus hendraense
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 178 - protein, 519 aa, unidentified
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 179 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 180 - protein, 15 aa, synthetic construct
TTAASGSSGGSSSGA

SEQ ID NO: 181 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 182 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 183 - DNA, 972 bp, Homo sapiens
  source 1..972: mol_type = genomic DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 184 - DNA, 918 bp, Homo sapiens
  source 1..918: mol_type = genomic DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 185 - DNA, 969 bp, synthetic construct
  source 1..969: mol_type = other DNA
atgtggcccctggtcgccgccctgttgctgggctcggcatgctgcggatcagctcagctactgtttaataaaacaaaatctgtagaattcacgttttgtaacgacactgtcgtgatcccatgctttgttactaatatggaggcacaaaacaccactgaagtgtacgtgaagtggaaattcaaaggcagagacatttacacctttgacggcgccctcaacaagtccaccgtgcccactgactttagtagcgcaaaaattgaggtcagccaattactaaaaggagatgcctctttgaagatggacaagagcgatgctgtcagccacacagggaactacacttgtgaagtaacagagttaacccgcgaaggtgaaacgatcatcgagctgaagtatcgagtggtgtcctggttttctccgaacgagaatatccttatcgtaattttcccaattttcgctatcctcctgttctggggccagtttggtatcaagacactcaaatatcggtccggtgggatggatgagaagacaattgccctgcttgttgctggactcgtgatcaccgtcatcgtgattgttggggccatccttttcgtcccaggggagtacagcctgaagaatgctacgggcctgggattaattgtgacctctacagggatactcatcctgcttcactactatgtgttcagtaccgcgattggactgacctccttcgtcattgccatattggtgattcaggtgatagcctacatcctcgccgtggttggcctgagtctctgtatcgcggcgtgcatacccatgcatggccctcttctgatttcagggttgagtatcctcgcactagcacagttgctgggactggtttatatgaaatttgtggcctccaaccagaagactatacagcctcctaggaaggctgtagaggagcccctgaatgcattcaaggaatcaaaaggcatgatgaatgatgaa

SEQ ID NO: 186 - protein, 21 aa, synthetic construct
  VARIANT 1..3: note = May be absent
GSGEGRGSLLTCGDVEENPGP

SEQ ID NO: 187 - protein, 22 aa, synthetic construct
  VARIANT 1..3: note = May be absent
GSGATNFSLLKQAGDVEENPGP

SEQ ID NO: 188 - protein, 23 aa, synthetic construct
  VARIANT 1..3: note = May be absent
GSGQCTNYALLKLAGDVESNPGP

SEQ ID NO: 189 - protein, 25 aa, synthetic construct
  VARIANT 1..3: note = May be absent
GSGVKQTLNFDLLKLAGDVESNPGP

SEQ ID NO: 190 - protein, 7 aa, Homo sapiens
  VARIANT 5..7: note = May be absent
RRRRGSG

SEQ ID NO: 191 - protein, 7 aa, Homo sapiens
  VARIANT 5..7: note = May be absent
RKRRGSG

SEQ ID NO: 192 - protein, 15 aa, Homo sapiens
  VARIANT 5..7: note = May be absent
  VARIANT 13..15: note = May be absent
RKRRGSGTPDPWGSG

SEQ ID NO: 193 - skipped

SEQ ID NO: 194 - RNA, 12 nt, synthetic construct
  source 1..12: mol_type = other RNA
gttttagagcta

SEQ ID NO: 195 - skipped

SEQ ID NO: 196 - RNA, 66 nt, synthetic construct
  source 1..66: mol_type = other RNA
tagcaagttaaaataaggctagtccgttatcaacttgaaaaagtggcaccgagtcggtgctttttt

SEQ ID NO: 197 - skipped

SEQ ID NO: 198 - RNA, 17 nt, synthetic construct
  source 1..17: mol_type = other RNA
gtttsagagctatgctg

SEQ ID NO: 199 - skipped

SEQ ID NO: 200 - RNA, 71 nt, synthetic construct
  source 1..71: mol_type = other RNA
cagcatagcaagttsaaataaggctagtccgttatcaacttgaaaaagtggcaccgagtcggtgctttttt

SEQ ID NO: 201 - skipped

SEQ ID NO: 202 - RNA, 14 nt, synthetic construct
  source 1..14: mol_type = other RNA
gttttagtactctg

SEQ ID NO: 203 - skipped

SEQ ID NO: 204 - RNA, 64 nt, synthetic construct
  source 1..64: mol_type = other RNA
cagaatctactaaaacaaggcaaaatgccgtgtttatctcgtcaacttgttggcgagatttttt

SEQ ID NO: 205 - RNA, 88 nt, synthetic construct
  source 1..88: mol_type = other RNA
gtcgtctataggacggcgaggacaacgggaagtgccaatgtgctctttccaagagcaaacaccccgttggcttcaagatgaccgctcg

SEQ ID NO: 206 - skipped

SEQ ID NO: 207 - RNA, 23 nt, synthetic construct
  source 1..23: mol_type = other RNA
cgagcggtctgagaagtggcact

SEQ ID NO: 208 - skipped

SEQ ID NO: 209 - protein, 7 aa, synthetic construct
GFTFSSY

SEQ ID NO: 210 - protein, 7 aa, synthetic construct
GFTFDDY

SEQ ID NO: 211 - protein, 7 aa, synthetic construct
SFDFDYY

SEQ ID NO: 212 - protein, 6 aa, synthetic construct
SGSGGS

SEQ ID NO: 213 - protein, 6 aa, synthetic construct
SWNSGS

SEQ ID NO: 214 - protein, 5 aa, synthetic construct
HYSGS

SEQ ID NO: 215 - protein, 6 aa, synthetic construct
SGSGDS

SEQ ID NO: 216 - protein, 11 aa, synthetic construct
ARVAMTGAFDI

SEQ ID NO: 217 - protein, 13 aa, synthetic construct
ARVVPAAYGAFDI

SEQ ID NO: 218 - protein, 11 aa, synthetic construct
VKVYQQGAFDI

SEQ ID NO: 219 - protein, 9 aa, synthetic construct
ARGQLRLDV

SEQ ID NO: 220 - protein, 13 aa, synthetic construct
ARATLAVAGASDY

SEQ ID NO: 221 - protein, 12 aa, synthetic construct
RASQSVSSSYLA

SEQ ID NO: 222 - protein, 11 aa, synthetic construct
RASQSISTYLN

SEQ ID NO: 223 - protein, 12 aa, synthetic construct
RLLIYGASSRAT

SEQ ID NO: 224 - protein, 12 aa, synthetic construct
KLLIHAASSLQG

SEQ ID NO: 225 - protein, 10 aa, synthetic construct
QQYSSSPGFT

SEQ ID NO: 226 - protein, 10 aa, synthetic construct
QHYGNPPLYT

SEQ ID NO: 227 - protein, 9 aa, synthetic construct
QQSLMTPLT

SEQ ID NO: 228 - protein, 10 aa, synthetic construct
QHYGGSPLFT

SEQ ID NO: 229 - protein, 8 aa, synthetic construct
GFTFSSYA

SEQ ID NO: 230 - protein, 8 aa, synthetic construct
GFTFDDYA

SEQ ID NO: 231 - protein, 8 aa, synthetic construct
SFDFDYYE

SEQ ID NO: 232 - protein, 8 aa, synthetic construct
ISGSGGST

SEQ ID NO: 233 - protein, 8 aa, synthetic construct
ISWNSGSI

SEQ ID NO: 234 - protein, 7 aa, synthetic construct
IHYSGST

SEQ ID NO: 235 - protein, 11 aa, synthetic construct
ARVAMTGAFDI

SEQ ID NO: 236 - protein, 13 aa, synthetic construct
ARVVPAAYGAFDI

SEQ ID NO: 237 - protein, 9 aa, synthetic construct
VKVYQQGDI

SEQ ID NO: 238 - protein, 11 aa, synthetic construct
ARGQLRLAFDV

SEQ ID NO: 239 - protein, 13 aa, synthetic construct
ARATLAVAGASDY

SEQ ID NO: 240 - protein, 7 aa, synthetic construct
QSVSSSY

SEQ ID NO: 241 - protein, 6 aa, synthetic construct
QSISTY

SEQ ID NO: 242-243 - skipped (2 entries)

SEQ ID NO: 244 - protein, 10 aa, synthetic construct
QQYSSSPGFT

SEQ ID NO: 245 - protein, 10 aa, synthetic construct
QHYGNPPLYT

SEQ ID NO: 246 - protein, 9 aa, synthetic construct
QQSLMTPLT

SEQ ID NO: 247 - protein, 10 aa, synthetic construct
QHYGGSPLFT

SEQ ID NO: 248-249 - skipped (2 entries)

SEQ ID NO: 250 - protein, 4 aa, synthetic construct
GGGS

SEQ ID NO: 251 - protein, 8 aa, synthetic construct
ISGSGDST

SEQ ID NO: 252 - protein, 30 aa, synthetic construct
  VARIANT 1..30: note = This sequence may encompass 1-10 GGS repeating units
GGSGGSGGSGGSGGSGGSGGSGGSGGSGGS